(19) United States
(12) Patent Application Publication
(10) Pub. No.: US 2016/0296145 A1
(43) Pub. Date: Oct. 13, 2016

(54) Wearable diagnostic platform using direct magnetic detection of magnetic nanoparticles in vivo

(71) Applicant: Verily Life Sciences LLC, Mountain View, CA (US)

(72) Inventors: Vikram Singh Bajaj, Mountain View, CA (US); Vasiliki Demas, San Jose, CA (US); Victor Marcel Acosta, Mountain View, CA (US); James Higbie, Palo Alto, CA (US); John David Perreault, Mountain View, CA (US)

(21) Appl. No.: 15/088,832

(22) Filed: Apr. 1, 2016

Related U.S. Application Data
(60) Provisional application No. 62/144,646, filed on Apr. 8, 2015.

(51) Int. Cl. A61B 5/145; A61B 5/05

(52) U.S. Cl. CPC A61B 5/145; A61B 5/05

(57) ABSTRACT

Wearable devices configured to detect the presence, concentration, number, or other properties of magnetic nanoparticles disposed in subsurface vasculature of a person are provided. The wearable devices are configured to detect, using one or more magnetometers, magnetic fields produced by the magnetic nanoparticles. In some embodiments, the magnetometer(s) are atomic magnetometers. In some embodiments, the wearable devices include magnets or other means to magnetize the magnetic nanoparticles. In some embodiments, the wearable devices produce a time-varying magnetic field, and the magnetometer(s) are configured to detect a time-varying magnetic field responsively produced by the magnetic nanoparticles. In some embodiments, the magnetic nanoparticles are configured to bind to an analyte of interest and detected properties of the magnetic nanoparticles can be used to determine the presence, concentration, or other properties of the analyte.

### CROSS-REFERENCE TO RELATED APPLICATION

This application claims priority to U.S. Provisional Patent Application No. 62/144,646, filed Apr. 8, 2015, which is incorporated herein by reference.

### BACKGROUND

Unless otherwise indicated herein, the materials described in this section are not prior art to the claims in this application and are not admitted to be prior art by inclusion in this section.

A number of scientific methods have been developed to detect, measure, and/or affect one or more analytes in a biological or other environment (e.g., a person's body). The one or more analytes could be any analytes that, when present in or absent from a person's body, or present at a particular concentration or range of concentrations, may be indicative of a medical condition or health state of the person. The one or more analytes could be substances whose distribution, action, or other properties, interactions, or activities throughout an animal's body is of scientific or medical interest. The one or more analytes could include pharmaceuticals or other substances introduced into the biological or other environment to effect some chemical or biological process. The one or more analytes could be present in living or nonliving human or animal tissue, and could be detected, measured, or affected in an in vivo, ex vivo, in vitro, or some other type of sample. The one or more analytes could include enzymes, reagents, hormones, proteins, drugs, nanoparticles, pharmaceuticals, cells or other molecules.

### SUMMARY

Some embodiments of the present disclosure provide a device including: (i) a magnetometer, wherein the magnetometer is configured to be positioned proximate to a biological environment, and wherein the magnetometer is configured to detect magnetic fields produced by magnetic nanoparticles in the biological environment that are proximate the magnetometer; and (ii) a controller operably coupled to the magnetometer, wherein the controller includes a computing device programmed to perform controller operations including: (a) operating the magnetometer to detect a magnetic field; and (b) determining a property of magnetic nanoparticles in the biological environment based on the detected magnetic field.

Some embodiments of the present disclosure provide a system including: (i) means for detecting a magnetic field proximate to a biological environment, wherein the means for detecting a magnetic field are configured to be positioned proximate to the biological environment, and wherein the means for detecting a magnetic field are configured to detect magnetic fields produced by magnetic nanoparticles in the biological environment that are proximate the means for detecting a magnetic field; and (ii) controller means operably coupled to the means for detecting a magnetic field, wherein the controller means include a computing device programmed to perform controller operations including: (a) operating the means for detecting a magnetic field to detect a magnetic field; and (b) determining a property of magnetic nanoparticles in the biological environment based on the detected magnetic field.

Some embodiments of the present disclosure provide a method including: (i) detecting, using a magnetometer, a magnetic field proximate to a biological environment, wherein detecting a magnetic field proximate to a biological environment includes detecting a magnetic field produced by magnetic nanoparticles in the biological environment that are proximate the magnetometer; and (ii) determining a property of magnetic nanoparticles in the biological environment based on the detected magnetic field.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1A is a side cross-sectional view of magnetic particles in a portion of subsurface vasculature and a device positioned proximate to the portion of subsurface vasculature, in accordance with an example embodiment.

FIG. 1B illustrates an example output over time of a magnetic sensor of the device of FIG. 1A as magnetic particles in the portion of subsurface vasculature of FIG. 1A move through the portion of subsurface vasculature.

FIG. 2A is a side cross-sectional view of magnetic particles in a portion of subsurface vasculature and a device positioned proximate to the portion of subsurface vasculature, in accordance with an example embodiment.

FIG. 2B illustrates example outputs over time of two magnetic sensors of the device of FIG. 2A as magnetic particles in the portion of subsurface vasculature of FIG. 2A move through the portion of subsurface vasculature.

FIG. 2C illustrates an example signal related to the motion of magnetic particles in the portion of subsurface vasculature of FIG. 2A based on the example outputs illustrated in FIG. 2B

FIG. 3A is a side cross-sectional view of magnetic particles in a portion of subsurface vasculature and a device positioned proximate to the portion of subsurface vasculature, in accordance with an example embodiment.

FIG. 3B illustrates an example output over time of a magnetic sensor of the device of FIG. 3A, an example magnetic field generated by a magnetic coil of the device of FIG. 3A, and an example magnetic field generated by magnetic particles in the portion of subsurface vasculature of FIG. 3A.

FIG. 4 illustrates an example frequency spectrum of an output of a magnetic sensor.

FIG. 5 is a side cross-sectional view of magnetic particles in a portion of subsurface vasculature and a device positioned proximate to the portion of subsurface vasculature, in accordance with an example embodiment.

FIG. 6A is a side cross-sectional view of magnetic particles in a portion of subsurface vasculature and a device positioned proximate to the portion of subsurface vasculature during a first period of time, in accordance with an example embodiment.

FIG. 6B is a side cross-sectional view of the magnetic particles in the portion of subsurface vasculature of FIG. 6A and the device positioned proximate to the portion of subsurface vasculature of FIG. 6A during a second period of time, in accordance with an example embodiment.

FIG. 7 is perspective view of an example device.

FIG. 8A is a perspective view of an example device mounted to a wrist of a wearer.

FIG. 8B is another perspective view of the example device of FIG. 8A.

FIG. 9 is an illustration of a number of wearable devices in communication with a server.

FIG. 10 is a block diagram of an example system.

FIG. 11 is a flowchart of an example method

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

### I. OVERVIEW

Magnetic particles can be configured to selectively bind with an analyte of interest. Magnetic particles configured in this way can enable manipulation of, detection of, or other interactions with the analytes by applying magnetic forces to the magnetic particles. Additionally or alternatively, an analyte of interest could be intrinsically magnetic, or could be an engineered analyte (e.g., a pharmaceutical) that includes a magnetic property and/or that is bound to a magnetic particle and that can be introduced into an environment according to an application. Detecting the magnetic field produced by such magnetic particles could allow for the determination of the amount (e.g., concentration, number), distribution, or other properties of the analyte of interest in the biological environment. For example, the magnetic field produced by such analyte-binding magnetic particles in a portion of subsurface vasculature could be detected (e.g., using one or more magnetometers disposed in a wearable device mounted proximate to the portion of subsurface vasculature) and used to determine the number and/or concentration of the analyte in the blood in the portion of subsurface vasculature.

Magnetic nanoparticles may be made of and/or wholly or partially coated by an inert material, such as polystyrene, and can have a diameter that is less than about 20 micrometers. In some embodiments, the particles have a diameter on the order of about 5 nm to 1 μm. In further embodiments, one or more magnetic nanoparticles may be embedded in a substrate of non-magnetic material (e.g., polystyrene). In some examples, the size and/or a distribution of sizes of such magnetic nanoparticles could be specified to control a magnetic or other property of the magnetic nanoparticles, e.g., to control a coercivity, remanence, type of magnetic behavior (e.g., superparamagnetism, ferromagnetism, ferrimagnetism), hysteresis, or other property of the magnetic nanoparticles. For example, a particle of magnetic material of a magnetic nanoparticle could have a size between approximately 10 nanometers and approximately 20 nanometers e.g., such that the magnetic nanoparticle comprises a single magnetic domain. The magnetic nanoparticles may be magnetic and can be formed from a paramagnetic, super-paramagnetic or ferromagnetic material or any other material that responds to a magnetic field.

Those of skill in the art will understand a “particle” in its broadest sense and that it may take the form of any fabricated material, a molecule, cryptophane, a virus, a phage, etc. Further, a magnetic nanoparticle may be of any shape, for example, spheres, rods, non-symmetrical shapes, etc. Further, the magnetic nanoparticles can be configured to selectively bind to one or more analytes (e.g., chemicals, hormones, peptides, DNA or RNA fragments, cells). Such particles could be introduced into an environment that contains the one or more analytes (e.g., into the blood of a body, into a portion of subsurface vasculature of a body, into a fluid of a natural environment, water treatment process, pharmaceutical process, or some other environment of interest). Alternatively, the one or more analytes and/or a fluid or other material containing the one or more analytes could be extracted (e.g., from an environment of interest) and introduced into another environment into which the magnetic nanoparticles have been or could be introduced.

Detection of magnetic fields produced by magnetic nanoparticles could provide a variety of applications. The magnetic nanoparticles could be configured to selectively interact with (e.g., to bind to) one or more analytes of interest. Detection of the magnetic fields produced by the magnetic nanoparticles could allow for the determination of one or more properties of the analytes of interest, e.g., a concentration of the analytes, a number of the analytes (e.g., a number of cancer cells in a portion of subsurface vasculature and/or in the blood circulation of a body), a property of the analytes, or some other information about the analytes. Detection of magnetic fields produced by magnetic nanoparticles could allow the determination of the orientation and/or location of the magnetic nanoparticles (e.g., by detecting a magnitude and/or direction of the produced magnetic field at one or more locations proximate to (e.g., outside of) the environment of interest, e.g., outside skin proximate a portion of subsurface vasculature), a degree of aggregation of the magnetic nanoparticles (e.g., by detecting a magnitude of the produced magnetic field, by detecting a property of change over time of the produced magnetic field), or the detection of some other property of the magnetic nanoparticles. Such determined properties of the magnetic nanoparticles could be related to properties of the analytes of interest. For example, multiple magnetic nanoparticles could bind to a single instance of an analyte (e.g., to a single cancer cell) such that detection of an aggregate of magnetic nanoparticles (e.g., detection of a large amplitude magnetic field produced by such aggregated magnetic nanoparticles) allows for the determination that the single instance of the analyte is present (e.g., that a cancer cell is present in a portion of subsurface vasculature). Other properties of a detected magnetic field produced by magnetic nanoparticles could be used in similar or different ways to determine properties of one or more analytes in an environment of interest.

One or more properties of the analyte could be related to a medical condition of a human or animal containing the analyte. In some examples, the analyte could have a medical or other effect on the human or animal (e.g., the analyte is a toxin, the analyte is a pharmaceutical, the analyte is a cancer cell), and detecting magnetic fields produced by magnetic nanoparticles bound to the analyte could allow detection or determination of a medical condition of the human or animal. For example, the analyte could be a cancer cell, and detection of the magnetic fields produced by magnetic nanoparticles in the blood could allow the detection of an amount of the cancer cells in the blood, a stage of the cancer, that the cancer has entered or left remission, or some other information or health state. In some examples, magnetic nanoparticles could be used to collect an analyte (e.g., by exerting a magnetic force to collect magnetic nanoparticles bound to the analyte), to control a rate of administration of a drug (e.g., by producing magnetic fields to manipulate magnetic nanoparticles bound to the drug), to modify or destroy an analyte (e.g., by applying RF energy to the magnetic particles such that analytes bound to the magnetic particles are modified or destroyed), or to provide some other function. Other applications and environments containing magnetic nanoparticles are anticipated.

A variety of properties of the magnetic field produced by magnetic nanoparticles could be detected in a variety of ways. A direction, magnitude, property of change over time, or some other property of the produced magnetic fields could be detected. Such detection could include operating one or more magnetometers (i.e., devices or components configured to detect one or more properties, e.g., magnitude, direction, magnitude in a specified direction, of a magnetic field) to detect produced magnetic fields at one or more respective locations proximate to (e.g., outside of) an environment of interest that contains the magnetic nanoparticles. For example, a body-mountable device including one or more magnetometers could be mounted to a skin surface proximate a portion of subsurface vasculature such that the one or more magnetometers can detect magnetic fields produced by the magnetic particles in the portion of subsurface vasculature. Such magnetometers could be configured to detect magnetic fields that have very small magnitudes. For example, a magnetometer used to detect magnetic fields produced by magnetic nanoparticles could be configured to have a sensitivity such that the magnetometer can detect changes in a measured magnetic field (e.g., a magnetic field at a location less than approximately 1 centimeter outside a portion of subsurface vasculature) of less than approximately 10 femtoteslas.

Magnetometers could include superconducting quantum interference devices (SQUIDs), spin-exchange relaxation-free (SERF) magnetometers, inductive loops or coils or other antenna structures, spin precession magnetometers, or some other magnetic-field-detecting components or devices. Further, the magnetic field produced by magnetic nanoparticles could be detected at more than one location (e.g., by more than one magnetometer) to allow for detection of properties of the magnetic nanoparticles (e.g., to detect a speed of movement in a portion of subsurface vasculature) and/or to allow a background magnetic field (e.g., a magnetic field in present in the environment of interest that is not produced by the magnetic nanoparticles, e.g., that is produced by the Earth, that is produced by electronic devices, that is produced by other magnetic and/or magnetized materials in or proximate to the environment of interest.

The magnetic nanoparticles could produce a magnetic field intrinsically, e.g., the magnetic nanoparticles could include magnetized ferromagnetic materials and/or the magnetic nanoparticles could include superparamagnetic materials that become spontaneously magnetized. In such examples, this intrinsically produced magnetic field could be detected (e.g., by a magnetometer) and used to determine one or more properties of an analyte to which the magnetic nanoparticles are configured to bind. Additionally or alternatively, the magnetic field produced by the magnetic nanoparticles could be induced by an external static and/or time-varying magnetic field or other applied energy or field. For example, a permanent magnet, electromagnet, or other magnetic field producing component could produce a magnetic field in an environment of interest (e.g., in a portion of subsurface vasculature) sufficient to magnetize the magnetic nanoparticles, and the magnetic field produced by the magnetized magnetic nanoparticles could be detected. In another example, an alternating (e.g., sinusoidal) magnetic field could be produced (e.g., by an electronically driven coil) in an environment of interest containing the magnetic nanoparticles, and magnetic fields reflected, phase-shifted, frequency-shifted, frequency-multiplied, or otherwise produced by the magnetic nanoparticles could be detected.

In some examples, one or more properties of the analyte could be determined and/or detected by collecting the magnetic nanoparticles such that a magnitude of the magnetic field produced by the magnetic particles and detected by a magnetometer is increased. Such collection could include producing a magnetic field in an environment of interest such that a magnetic force is exerted on the magnetic nanoparticles to collect the magnetic nanoparticles. In some examples, an electromagnet, permanent magnet, or other magnetic field-producing component could be operated to collect the magnetic nanoparticles and subsequently to release the collected magnetic nanoparticles (e.g., to provide detection of a magnetic field produced by the magnetic nanoparticles without interference by the magnetic field produced by the electromagnet, permanent magnet, or other magnetic field-producing component).

The effects of a background magnetic field (e.g., a magnetic field produced by electronics or magnetic materials proximate to and/or within an environment of interest, a magnetic field produced by the Earth) could be mitigated or compensated for in a variety of ways. In some examples, a system could include two or more magnetometers configured to detect magnetic fields at two or more respective locations. In such examples, a magnetic field produced by magnetic particles in the environment of interest could be determined by determining a difference between the magnetic fields detected by two of the two or more magnetometers. In some examples, a system could include magnetic shims, magnetic shielding materials, permanent magnets, electromagnets, or other means for changing and/or controlling a magnetic field detected by a magnetometer. Such means could be used to reduce a background magnetic field detected at a location by the magnetometer (e.g., to cancel a magnetic field produced by the Earth and detected by the magnetometer) and/or to cancel a magnetic field produced by a component of the system or by some other system (e.g., a magnetic field produced by an electromagnet to magnetize and/or collect magnetic nanoparticles). Such means could be operated based on a magnetic field detected by the magnetometer (e.g., to zero the output of the magnetometer), based on a magnetic field detected by another magnetometer (e.g., to reduce the magnetic field present at the location of a SERF magnetometer based on a magnetic field detected by a hall effect magnetometer located proximate to the SERF magnetometer), or based on some other information or consideration.

Magnetometers configured as described herein could be included as part of a variety of systems or devices and configured to detect magnetic fields produced by magnetic nanoparticles present in a variety of environments according to a variety of applications. In some examples, one or more magnetometers or other components could be included in a body-mountable device configured to be mounted to a skin surface and to detect magnetic fields produced by magnetic nanoparticles in a portion of subsurface vasculature proximate the skin surface. Additionally or alternatively, magnetometers configured to detect magnetic fields produced by magnetic nanoparticles could be included in handhelds, desktop, wall- or floor-mounted devices, or some other type of device or system. Such systems could be configured to detect magnetic fields produced by magnetic nanoparticles disposed in natural environments (e.g., portions of subsurface vasculature, fluids of a lake, stream, or other natural outdoor environment), ex vivo and/or in vitro environments (e.g., fluids contained in a sample container), artificial environments (e.g., a fluid or other volume of a pharmaceutical or industrial process), or some other environment of interest. Magnetic nanoparticles could be disposed in a flowing fluid or otherwise moving environment and/or disposed in a substantially static fluid or otherwise nonmoving environment. Magnetic nanoparticles could be introduced into the environment of interest (e.g., injected into a portion of subsurface vasculature), naturally present in the environment of interest, introduced into a sample extracted from an environment of interest, or otherwise disposed relative to an environment of interest.

It should be understood that the above embodiments, and other embodiments described herein, are provided for explanatory purposes, and are not intended to be limiting.

Further, the term “medical condition” as used herein should be understood broadly to include any disease, illness, disorder, injury, condition or impairment—e.g., physiologic, psychological, cardiac, vascular, orthopedic, visual, speech, or hearing—or any situation requiring medical attention.

### II. ILLUSTRATIVE MAGNETIC PARTICLES AND DETECTION OF MAGNETIC FIELDS THEREOF

Magnetic fields produced by magnetic nanoparticles in an environment of interest can be detected (e.g., by one or more magnetometers located within and/or proximate to the environment of interest) and used to determine the location, amount (e.g., number, concentration), orientation, velocity, degree of aggregation, or other properties of the magnetic nanoparticles in the environment of interest and/or to determine properties of the environment of interest. The environment of interest could include artificial environments (e.g., a fluid of an industrial process, a fluid of a chemical or pharmaceutical process) or natural environments (e.g., a lake, a river, a marsh, blood in vasculature of an animal). For example, the magnetic nanoparticles could be disposed in blood in a portion of subsurface vasculature of a human. The magnetic nanoparticles could be permanently magnetized (e.g., could be ferromagnetic) or could become magnetized when exposed to a magnetic field (e.g., could be paramagnetic, superparamagnetic) or to some other factor. In some examples, the magnetic nanoparticles can be configured to bind to an analyte of interest and magnetic fields produced by the magnetic nanoparticles could be detected to determine the location, amount (e.g., number, concentration), state of binding to one or more magnetic nanoparticles, or other properties of the analyte of interest.

The magnetic field produced by one or more magnetic nanoparticles can be detected at one or more locations in space. The direction, magnitude, and/or other properties of the produced magnetic field at a particular location can be related to the location and/or orientation of the one or more magnetic nanoparticle relative to the particular location, the magnitude of the permanent and/or induced magnetic dipole moment of the magnetic nanoparticle, magnetic properties of materials proximate the particular location, or other factors. A magnetic field at the particular location (e.g., a direction and/or magnitude of a magnetic field detected by, e.g., a magnetometer) could be related to the magnetic field of the earth, magnetic fields produced by electronics or other devices proximate the particular location, magnetized or otherwise magnetic materials proximate the particular location, or other factors in addition to the magnetic field produced by the one or more magnetic nanoparticles.

The magnetic nanoparticles could produce a magnetic field intrinsically, e.g., each magnetic nanoparticle could include magnetized ferromagnetic materials and/or each magnetic nanoparticle could include superparamagnetic materials that become spontaneously magnetized. In such examples, this produced intrinsic magnetic field could be detected at one or more locations (e.g., by a magnetometer) and used to determine one or more properties of the magnetic nanoparticles. For example, detecting a magnetic field (e.g., detecting a magnitude, direction, change over time, or other properties of the magnetic field) at a particular location could provide information about the location, orientation, number, state of binding to an analyte, degree of magnetization or other magnetic state, or some other information about magnetic nanoparticles proximate the particular location. Additionally or alternatively, the magnetic field produced by the magnetic nanoparticles could be induced by an external static and/or time-varying magnetic field or other applied energy or field. The magnetic nanoparticles could include a coating and/or be composed of a material that is biocompatible and/or specified to interact in some way with biological and/or chemical elements in an environment of interest (e.g., to interact specifically with an analyte of interest).

The magnetic nanoparticles may each include magnetic materials having a coercivity, remanence, magnetic moment, or other magnetic property such that the magnetic nanoparticles can produce a magnetic field (e.g., by being magnetized, by reflecting or otherwise interacting with a time-varying electromagnetic field) that could be detected by a magnetometer proximate to the magnetic nanoparticles. In some examples, this could include the magnetic nanoparticles each including a single piece of magnetic material, e.g., a single particle or crystal of a ferromagnetic, paramagnetic, superparamagnetic, or otherwise magnetic material. Such a magnetic material of a magnetic nanoparticle could be coated by an inert material, such as polystyrene. The magnetic nanoparticles could be similar (e.g., could each be similarly sized) or could vary, e.g., the size of the magnetic nanoparticles or some other properties of the magnetic nanoparticles could vary according to a distribution.

The magnetic nanoparticles could have an overall size and/or shape specified according to an application. For example, the magnetic nanoparticles could have a size and/or shape such that the magnetic nanoparticles can be transported in blood in the vasculature of a body without causing blockages and/or such that the magnetic nanoparticles produce a magnetic field having a sufficiently high magnitude to be detected by one or more magnetometers proximate the magnetic nanoparticles (e.g., to be detect by a magnetometer located outside of a portion of subsurface vasculature containing the magnetic nanoparticles, e.g., from approximately a millimeter to approximately a centimeter away from the magnetic nanoparticles). In some examples, the magnetic nanoparticles can have a diameter that is less than about 20 micrometers. In some embodiments, the magnetic nanoparticles particles have a diameter on the order of about 5 nm to 1 μm.

In further embodiments, magnetic nanoparticles and/to other small particles on the order of 10-100 nm in diameter may be assembled to form larger “clusters” or “assemblies” on the order of 1-10 micrometers. Those of skill in the art will understand a “particle” in its broadest sense and that it may take the form of any fabricated material, a molecule, cryptophan, a virus, a phage, etc. Further, a magnetic nanoparticle may be of any shape, for example, spheres, rods, non-symmetrical shapes, etc. In some examples, a magnetic material of the magnetic nanoparticles can include a paramagnetic, super-paramagnetic or ferromagnetic material or any other material that responds to a magnetic field. In some examples, the magnetic nanoparticles can include a magnetic moiety (e.g., an organic molecule that has a magnetic and/or magnetizable molecular orbital). Further, the particles can be configured to selectively bind to one or more analytes (e.g., chemicals, hormones, peptides, DNA or RNA fragments, cells). In some examples, the magnetic nanoparticles could be considered to include other elements (e.g., analytes, other magnetic or non-magnetic particles) bound to the magnetic nanoparticles. Other embodiments of magnetic nanoparticles are anticipated.

In some examples, the magnetic nanoparticles are functionalized to selectively interact with an analyte of interest. The magnetic nanoparticles can be functionalized by covalently attaching a bioreceptor designed to selectively bind or otherwise recognize a particular analyte (e.g., a clinically-relevant analyte, e.g., a cancer cell). For example, magnetic nanoparticles may be functionalized with a variety of bioreceptors, including antibodies, nucleic acids (DNA, siRNA), low molecular weight ligands (folic acid, thiamine, dimercaptosuccinic acid), peptides (RGD, LHRD, antigenic peptides, internalization peptides), proteins (BSA, transferrin, antibodies, lectins, cytokines, fibrinogen, thrombin), polysaccharides (hyaluronic acid, chitosan, dextran, oligosaccharides, heparin), polyunsaturated fatty acids (palmitic acid, phospholipids), or plasmids. The functionalized magnetic nanoparticles can be introduced into a portion of subsurface vasculature of a person or other environment of interest by injection, ingestion, inhalation, transdermal application, or in some other manner.

A clinically-relevant analyte could be any substance that, when present in the blood of a person or animal, or present at a particular concentration or range of concentrations and/or in a certain amount, may be indicative and/or causative of an adverse medical condition. For example, the clinically-relevant analyte could be an enzyme, hormone, protein, other molecule, or even whole or partial cells. In one relevant example, certain proteins have been implicated as a partial cause of Parkinson's disease. Thus, the development of Parkinson's disease might be prevented or retarded by providing magnetic nanoparticles functionalized with a bioreceptor that will selectively bind to this target. A magnetic field produced by the magnetic nanoparticles may then be detected, using one or more systems or devices as described herein (e.g., a magnetometer in a wearable device mounted to an external body surface proximate to a portion of subsurface vasculature), to detect a property (e.g., a concentration, a presence) of the bound protein (e.g., to inform a treatment, to adjust a dosage of a drug). As a further example, the analyte could be a cancer cell. By detecting magnetic field produced by magnetic particles configured to selectively interact with the cancer cells, the progress of cancer (e.g., remission, stage) may be quantified and used to inform some treatment or other action (e.g., to begin chemotherapy, to set a dosage of a chemotherapy drug).

In some examples, magnetic nanoparticles configured to selectively interact with (e.g., bind to) an analyte of interest could be used to provide some additional applications. For example, an attractive magnetic force could be applied to the magnetic nanoparticles to collect, extract, or otherwise manipulate the analyte. Additionally or alternatively, the magnetic nanoparticles could be used to modify or destroy the analyte of interest, e.g., by transducing an electromagnetic energy directed toward the magnetic nanoparticles (e.g., RF energy) into heat to denature or otherwise modify or destroy the analyte. In some examples, such operations (e.g., emission of an optical, RF, thermal, acoustical, or other type of energy to modify or destroy an analyte of interest) could be performed in response to determining some information about the analyte (e.g., determining that an instance of the analyte is proximate to a magnetometer of a device, and further within an area of effect of an energy emitter of the device) based on a detected magnetic field produced by the magnetic nanoparticles.

Magnetic fields produced by magnetic nanoparticles and detected at one or more locations (e.g., by magnetometers disposed at the one or more locations) can be used in a variety of ways to detect properties of the magnetic nanoparticles and/or to detect properties of an analyte of interest with which the magnetic nanoparticles are configured to selectively interact. For example, a direction, velocity, orientation, angular velocity, magnetic moment, degree of magnetization, or other properties of one or more magnetic nanoparticles could be determined based on a magnetic field detected at one or more locations. Further, the presence, concentration, location, velocity, or other properties of the analyte could be determined based on the detected magnetic field and/or based on the determined properties of the magnetic nanoparticles. For example, the magnetic nanoparticles could be configured such that a plurality of magnetic nanoparticles could selectively interact with (e.g., bind to) a single instance of the analyte of interest. In such examples, the detection and/or determination that a plurality of the magnetic nanoparticles are aggregated (e.g., proximate each other) could be used to determine that an instance of the analyte is located proximate the aggregated magnetic nanoparticles. Other properties of a detected magnetic field and/or determined properties of the magnetic nanoparticles could be used to determine properties (e.g., location, number, concentration) of the analyte. For example, a velocity, angular velocity, magnetic property, or other property of the magnetic nanoparticles could be related to interaction between the magnetic nanoparticles and the analyte.

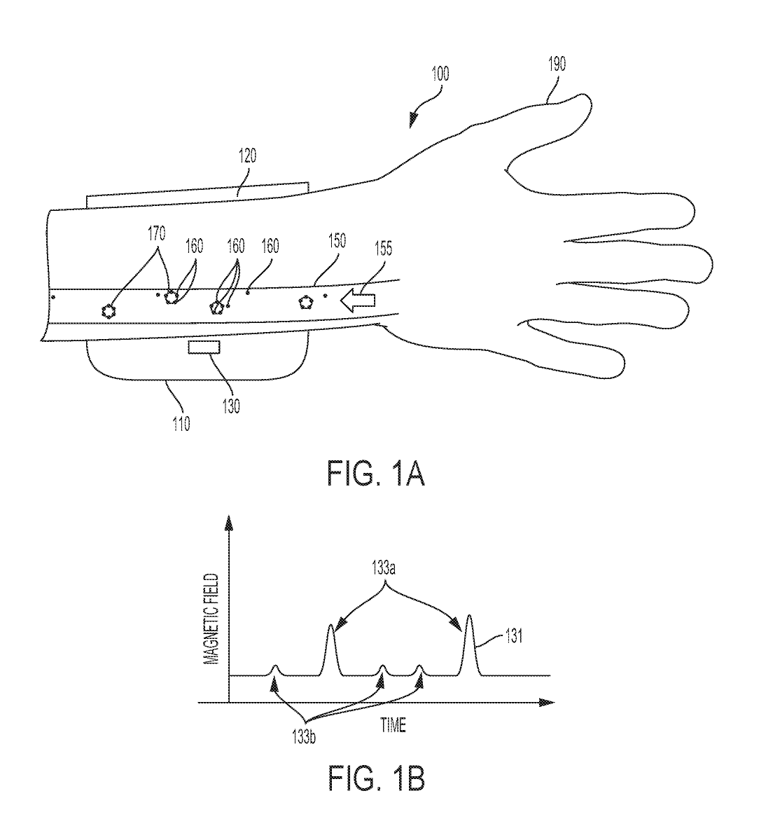

FIG. 1A illustrates example magnetic particles 160 and an analyte of interest 170 with which the magnetic particles 160 are configured to selectively interact disposed in a blood vessel 150 (i.e., a portion of subsurface vasculature). The blood vessel 150 is located in an arm 190 and contains blood that is flowing (direction of flow indicated by the arrow 155). A body-mountable device 100 includes a housing 110 mounted outside of or otherwise proximate to the blood vessel 150 by a mount 120 configured to encircle the arm 190. The body-mountable device 100 includes a magnetometer 130 disposed in the housing 110 and configured to detect a magnetic field at a location outside of the arm 190 (e.g., at a location within the magnetometer 130). The magnetic field detected by the magnetometer 130 could include magnetic fields produced by the magnetic nanoparticles 160 that are proximate the magnetometer 130, a magnetic field produced by the Earth, a magnetic field produced by electronics and/or electrical wiring (e.g., a magnetic field produced by an electromagnet, by other electronics of the body-mountable device 100, a magnetic field produced by a nearby automobile), a magnetic field produced and/or affected by a magnet or other magnetic material, and or some other magnetic fields and/or combinations of magnetic fields.

The analyte 170 and magnetic nanoparticles 160 are configured and distributed in the blood vessel 150 such that multiple magnetic nanoparticle 160 can bind to a single instance of the analyte 170 (e.g., to a single cancer cell). Further, magnetic nanoparticles 160 that are not bound to the analyte 170 are generally singly distributed throughout the blood in the blood vessel 150. As a result, the existence of an aggregate of magnetic nanoparticles 160 located proximate to each other could be related to the presence of one or more instances of the analyte 170 proximate the aggregate. Additionally or alternatively, the velocity, angular velocity, magnetic properties (e.g., magnetic moment, coercivity, type of magnetic behavior (e.g., ferromagnetism, paramagnetism, superparamagnetism)), or other properties of the magnetic particles 160 could be related to binding to the analyte 170 and/or to some other properties of the analyte 170, magnetic nanoparticles 160, and/or the blood vessel 150.

The magnetometer 130 could be configured to detect the magnitude, direction, magnitude parallel to a specified direction, frequency, rate of change, or other properties of the magnetic field at a particular location. The particular location could be a location on or within the magnetometer. The particular location could be a volume of space within the magnetometer, e.g., the magnetometer could be configured to detect the average magnitude of the magnetic field across a sensing volume within the magnetometer (e.g., a sensing volume that contains a high-temperature, high-density gas of alkali metal atoms that is optically interrogated by the magnetometer). The magnetometer could be configured to detect the magnetic field with a specified sensitivity such that the magnetometer can detect magnetic fields produced by the magnetic nanoparticles 160 proximate the magnetometer (e.g., magnetic nanoparticle located less than approximately 1 centimeter from a sensing volume of the magnetometer). For example, the magnetometer could have a sensitivity that is less than approximately 10 femtoteslas.

FIG. 1B illustrates an example signal 131 detected by the magnetometer 130 over time. The signal 131 represents the magnitude of the magnetic field over time. As shown in FIG. 1B, the signal 131 includes a number of pulses 133a, 133b related to respective increases in the magnetic field detected by the magnetometer. These pulses are related to the flow of blood 155 in the blood vessel 150 causing one or more magnetic nanoparticles 160 (e.g., single magnetic nanoparticles, aggregates of magnetic nanoparticles bound to the analyte 170) to become proximate to the magnetometer 130 (e.g., to become sufficiently proximate that the magnetic field produced by the one or more magnetic nanoparticles can be detected by the magnetometer 130) and subsequently to move away from the magnetometer 130.

The signal 131 includes lower-amplitude pulses 133b corresponding to the motion of individual magnetic nanoparticles 160 (e.g., magnetic nanoparticles that are not bound to the analyte 170) through the blood vessel 150 proximate the magnetometer 130. The signal 131 additionally includes higher-magnitude pulses 133a corresponding to the motion of aggregates of magnetic nanoparticles 160 (e.g., the aggregates may include magnetic nanoparticles bound to the analyte 170) through the blood vessel 150 proximate the magnetometer 130. The body-mountable device 100 could determine and/or detect the presence or other properties of the analyte 170 and/or of the magnetic nanoparticles 160 in the blood vessel 150 based on the width, amplitude, timing, or other properties of the detected pulses 133a, 133b. For example, a number of magnetic nanoparticles 160 proximate the magnetometer 130 at a particular time corresponding to a particular pulse detected in the signal 131 could be determined based on the amplitude of the particular pulse. For example, it could be determined that a single magnetic nanoparticle 160 is proximate to the magnetometer 130 at points in time corresponding to the lower-amplitude pulses 133b and that a plurality of magnetic nanoparticles 160 are proximate to the magnetometer 130 at points in time corresponding to the higher-amplitude pulses 133a. Related to this, it could be determined that an instance of the analyte 170 (e.g., a cancer cell) is proximate to the magnetometer at particular points in time corresponding to the higher-amplitude pulses 133a (e.g., related to the aggregation of the magnetic nanoparticles 160 by the analyte 170 causing an increase in the amplitude of the detected magnetic field).

Further, a size, number, or other properties of the analyte 170 could be determined based on the amplitude, width, shape, or other properties of the higher-amplitude pulses 133a and/or based on some other property of the detected magnetic field. For example, an amplitude of a pulse in the detected magnetic field could be related to a surface area of an instance of the analyte 170 (e.g., a greater surface area could permit more magnetic nanoparticles 160 to bind to the instance of analyte 170) and/or a number of instances of the analyte. A amount of the analyte 170 (e.g., a concentration of the analyte, a number of instances of the analyte) in a body could be determined based on a rate of detection of instances of the analyte (e.g., a rate of higher-amplitude pulses in the detected magnetic field), a mass flow rate of blood in the blood vessel 150, and/or other factors. A velocity of the analyte 170 and/or magnetic nanoparticles 160 could be related to a width of pulses in the detected magnetic field. Other properties of the analyte 170, the magnetic nanoparticles 160, the blood vessel 150, and/or the arm 190 could be detected and/or determined based on other features of a magnetic field detected using the magnetometer 130.

The signal 131 could represent the magnitude of the magnetic field detected by the magnetometer 130, the magnitude of the detected magnetic field in a particular direction, the amplitude or intensity of a time-varying (e.g., oscillating) magnetic field, the amplitude or intensity of a time-varying magnetic field within a range of frequencies, or some other detected and/or determined property of a magnetic field detected by the magnetometer 130. Further, a detected and/or determined property of the detected magnetic field over time could be similar or different from the illustrated example signal 131. Binding of the magnetic nanoparticles 160 to instances of the analyte 170 could be determined and/or detected based on other detected properties of the magnetic field produced by the magnetic nanoparticles 160 and/or by additional or alternative features thereof. For example, a velocity, an angular velocity, or some other property of motion of one or more magnetic nanoparticles 160 could be related to whether the magnetic nanoparticle is bound to one or more instances of the analyte 170. That is, magnetic nanoparticles 160 bound to the analyte 170 could be hindered from rotating by the analyte 170, could be sped or slowed in the flow 155 of blood in the blood vessel 150 by the analyte 170 (e.g., due to a drag coefficient of the analyte 170), or could exhibit some other property or behavior that is related to binding to the analyte 170 and that can be detected using the magnetometer 130.

Note that the use of the magnetometer 130 to detect magnetic fields produced by magnetic nanoparticles 160 in a flow 155 of blood in a blood vessel 150 and further to determine properties of the magnetic nanoparticles 160 and/or an analyte 170 to which the magnetic nanoparticles 160 are configured to bind is intended as a non-limiting illustrative example of embodiments described herein. Magnetic nanoparticles could be disposed in a variety of different environments (e.g., other bodily fluids, fluids of an animal, fluids of a natural environment, fluids of a medical, scientific, or industrial process). The magnetic nanoparticles could be disposed in a flowing fluid or in a substantially static fluid. The embodiments herein could be applied to the detection and/or determination of properties of magnetic nanoparticles and/or analytes in an ex vivo and/or in vitro flow cytometry experiment or process. One or more magnetometers configured to detect magnetic fields produced by magnetic nanoparticles could be disposed in a wearable, body-mountable, handheld, desktop, floor-, wall-, ceiling-, or otherwise-mounted, or otherwise configured device or system. Other environments and applications are anticipated.

In some examples, multiple magnetometers could be operated to detect magnetic fields produced by magnetic nanoparticles proximate the multiple magnetometers to provide applications described herein. Such multiple magnetometers could be configured and/or operated to detect a magnetic field gradient, to map a magnetic field across an area and/or volume, to determine a magnetic field produced by magnetic nanoparticles in an environment by detecting a magnetic field using a first magnetometer and subtracting a background magnetic field detected by a second magnetometer, or according to some other scheme to provide some other application(s).

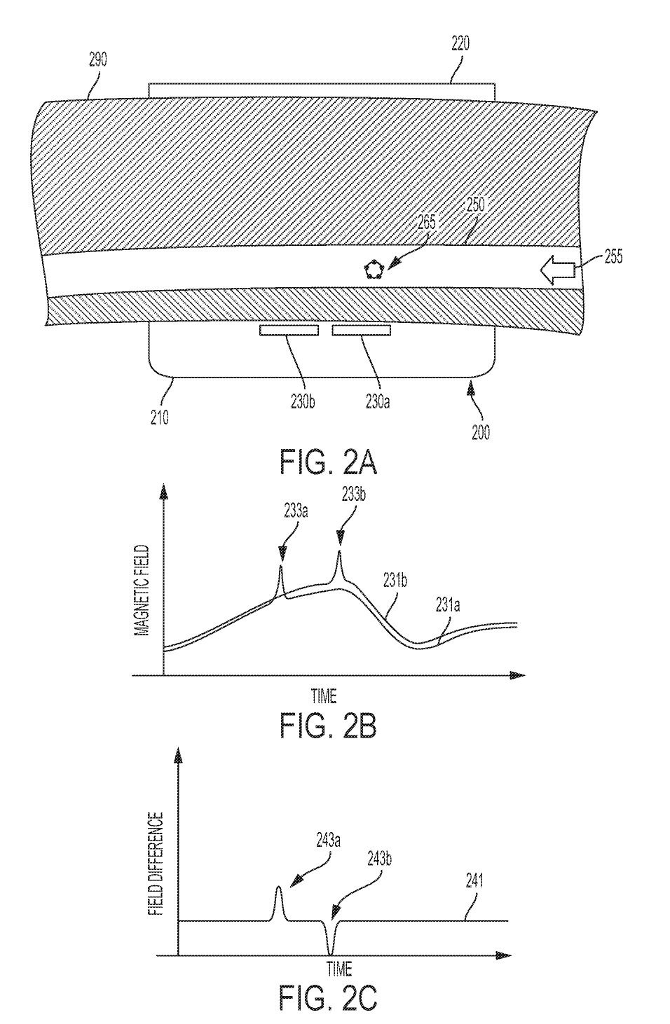

FIG. 2A illustrates an example complex 265 that includes magnetic particles bound to an analyte of interest disposed in a blood vessel 250 (i.e., a portion of subsurface vasculature). The blood vessel 250 is located in an arm 290 and contains blood that is flowing (direction of flow indicated by the arrow 255). A body-mountable device 200 includes a housing 210 mounted outside of the blood vessel 250 by a mount 220 configured to encircle the arm 290. The body-mountable device 200 includes first 230a and second 230b magnetometers disposed in the housing 210 and configured to detect magnetic fields at respective locations Proximate to (e.g., outside of) the arm 290 (e.g., at locations within the magnetometers 230a. 230b). The magnetic fields detected by the magnetometers 230a, 230b could include magnetic fields produced by magnetic nanoparticles of the complex 265 that are proximate the magnetometers 230a, 230b, a magnetic field produced by the Earth, a magnetic field produced by electronics and/or electrical wiring (e.g., a magnetic field produced by an electromagnet, by other electronics of the body-mountable device 200, a magnetic field produced by a nearby automobile), a magnetic field produced and/or affected by a magnet or other magnetic material, and or some other magnetic fields and/or combinations of magnetic fields.

FIG. 2B illustrates first 233a and second 233b example signals detected by the first 230a and second 230b magnetometers, respectively, over time. The signals 231a, 231b represent the magnitude of respective detected magnetic fields over time. As shown in FIG. 2B, the signals 231a, 231b each include a respective pulse 233a, 233b related to respective increases in the magnetic fields detected by the magnetometers 230a, 230b. These pulses are related to the flow of blood 255 in the blood vessel 250 causing the complex 265 to become proximate to each of the magnetometers 230a, 230b (e.g., to become sufficiently proximate that the magnetic field produced by the magnetic nanoparticles of the complex 265 can be detected by the magnetometers 230a, 230b) and subsequently to move away from the magnetometers 230a, 230b.

The signals 231a, 231b include a background signal substantially in common that corresponds to a background magnetic field detected by both of the magnetometers 230a, 230b. The signals 231a, 231b additionally include pulses 233a, 233b corresponding to the motion of the complex 265 through the blood vessel 250 proximate the first 230a and second 230b magnetometers, respectively. The body-mountable device 200 could determine the background magnetic field and/or determine the magnetic field produced by magnetic nanoparticles in the blood vessel 250 (e.g., 275) at the location of each of the magnetometers 230a, 230b based on the first 231a and second 231b signals. This could include performing a linear operation (e.g., averaging, subtraction, correlation, filtering), a nonlinear operation (e.g., nonlinear filtering, application of some probabilistic or clustering algorithm), or some other operation on one or both of the signals 231a, 231b. For example, FIG. 2C shows an example difference signal 241 determined as the difference between the first 231a and second 231b detected magnetic field signals. The difference signal 241 includes first 243a and second 243b pulses corresponding to the motion of the complex 265 through the blood vessel 250 proximate the first 230a and second 230b magnetometers, respectively.

The body-mountable device 200 could determine and/or detect the presence or other properties of the complex 265 and/or of an analyte and/or magnetic nanoparticles in the blood vessel 250 based on the width, amplitude, timing, or other properties of the detected pulses 233a, 233b. For example, a number of magnetic nanoparticles proximate a particular magnetometer (e.g., 230a, 230b) at a particular time corresponding to a particular pulse detected in the difference signal 241 could be determined based on the amplitude and/or sign of the particular pulse. For example, it could be determined that an aggregate of magnetic nanoparticles (e.g., 265) is proximate to the first magnetometer 230a at a point in time corresponding to the first, positive-sign pulse 243a. Further, a velocity of the complex 265 (or of some other magnetic element(s) producing magnetic fields detected by the magnetometers 230a, 230b) could be determined based on a difference in timing of detected pulses or other features of detected magnetic field signals produced by two or more magnetometers (e.g., 230a, 230b).

The magnetometers 230a, 230b could be configured to detect the same property of magnetic fields at respective locations (e.g., field magnitude, field magnitude in a specified direction, field direction) or different properties. The magnetometers could be similarly configured and/or the same type of magnetometer (e.g., the magnetometers 230a, 230b could both be SERF magnetometers, inductive pickup coils, SQUIDs) or differently configured. For example, the first magnetometer 230a could be less sensitive than the second magnetometer 230b and the output of the first magnetometer 230a could be used to operate the second magnetometer 230b (e.g., to set a bias, to set an offset, to apply a biasing magnetic field, or to otherwise improve the sensitivity or some other aspect of the operation of the second magnetometer 230b based on information about the magnetic field expected to be detected by the second magnetometer 230b determined from magnetic field information detected by the first magnetometer 230a).

Note that the background magnetic field detected by both magnetometers 230a, 230b and present substantially in common in both detected magnetic field signals 231a, 231b could be produced by and/or related to a variety of factors and/or objects. The background magnetic fields detected by the magnetometers 230a, 230b could include magnetic fields produced by magnetic elements and/or currents in the arm 290 that are substantially equally proximate to both magnetometers 230a, 230b, a magnetic field produced by the Earth, a magnetic field produced by electronics and/or electrical wiring (e.g., a magnetic field produced by an electromagnet, by other electronics of the body-mountable device 200, a magnetic field produced by a nearby automobile), a magnetic field produced and/or affected by a magnet or other magnetic material, and or some other magnetic fields and/or combinations of magnetic fields. As described herein, such a detected background magnetic field could be used to determine and/or detect the magnetic field produced by magnetic nanoparticles in a portion of subsurface vasculature (or other environment of interest), to operate one or more magnetometers (e.g., to set a bias, to apply a biasing magnetic field), or to provide some other operation related to the detection of magnetic fields produced by magnetic nanoparticles. Additionally or alternatively, such a detected background magnetic field could be used for some other application, e.g., to determine a local environmental magnetic field (e.g., related to magnetic north), to detect the location and/or orientation or changes of the body-mountable device 200 and/or changes thereof (e.g., motion, rotation), or to provide some other application.

In some examples, a detected and/or determined background field at a particular location could be reduced to improve the operation of a magnetometer to detect a magnetic field of interest (e.g., a magnetic field produced by magnetic nanoparticles proximate the location) at the particular location. This could be performed to reduce a dynamic range required to detect a magnetic field of interest, because a magnetometer is configured to operate in low-field conditions (e.g., the magnetometer is a SERF magnetometer configured to operate in magnetic fields less than some maximum value), or according to some other consideration. In some examples, this could include disposing magnetic shielding and/or shimming materials or components (e.g., components composed of mu-metal, ferrites, conductors, or other magnetic materials) to reduce the effect and/or presence of the background magnetic field at the particular location. In some examples, a biasing magnetic field could be applied to the particular location to cancel the background field. This could include magnets and/or electromagnets configured to provide the cancelling field. In some examples, the cancelling field could be controlled to match the background magnetic field, e.g., by controlling a location and/or orientation of a magnet and/or magnetic material (e.g., shim), by controlling a current applied to an electromagnetic coil, or by some other means.

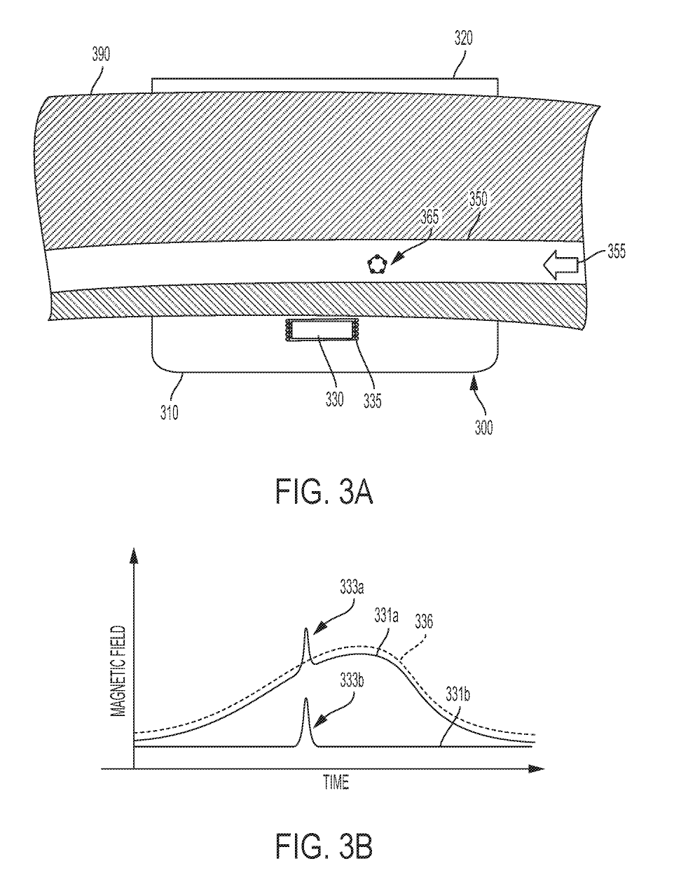

FIG. 3A illustrates an example complex 365 that includes magnetic particles bound to an analyte of interest disposed in a blood vessel 350 (i.e., a portion of subsurface vasculature). The blood vessel 350 is located in an arm 390 and contains blood that is flowing (direction of flow indicated by the arrow 355). A body-mountable device 300 includes a housing 310 mounted outside of the blood vessel 350 by a mount 320 configured to encircle the arm 390. The body-mountable device 300 includes a magnetometer 330 disposed in the housing 310 and configured to detect magnetic fields at a location proximate to (e.g., outside of) the arm 390 (e.g., at a location within the magnetometer 330). The body-mountable device 300 additionally includes a bias coil 335 disposed proximate to the magnetometer 330 and configured to produce a bias magnetic field such that the magnetic field detected by the magnetometer 330 (e.g., the magnetic field at the location outside of the arm 390) is reduced by an amount related to the bias magnetic field (e.g., the detected magnetic field is substantially equal to the vector sum of the bias magnetic field and any other magnetic fields present at the location outside the arm, e.g., a magnetic field produced by the complex 365). The magnetic fields detected by the magnetometer 330 could additionally include a magnetic field produced by the Earth, a magnetic field produced by electronics and/or electrical wiring (e.g., a magnetic field produced by an electromagnet, by other electronics of the body-mountable device 300, a magnetic field produced by a nearby automobile), a magnetic field produced and/or affected by a magnet or other magnetic material, and or some other magnetic fields and/or combinations of magnetic fields.

FIG. 3B illustrates an unbiased magnetic field 331a that could be present at the location at which the magnetometer detects a magnetic field over time. The unbiased magnetic field 331a includes a pulse 333a related to an increases in the magnetic field detected by the magnetometer 330 related to the flow of blood 355 in the blood vessel 350 causing the complex 365 to become proximate to the magnetometer 330 and subsequently to move away from the magnetometer 330. FIG. 3B additionally illustrates a bias field magnitude 336 that shows the magnitude of the bias field generated by the bias coil 335 over time, as measured at the location at which the magnetometer detects a magnetic field. The bias magnetic field (i.e., the magnetic field generated by the bias coil 335 according to the bias field magnitude 336) and the unbiased magnetic field (i.e., the magnetic fields present at the location at which the magnetometer detects a magnetic field that are not produced by the bias coil 335) have opposite directions at the location at which the magnetometer detects a magnetic field; that is, the bias magnetic field at least partially cancels the unbiased magnetic field. The bias field magnitude 336 could be determined in a number of ways, e.g., based on magnetic field values detected by the magnetometer 330 at previous points in time, on a magnetic field detected by another magnetometer (not shown), on the output of some other sensor (not shown), or based on some other consideration. FIG. 3B further illustrates an example detected signal 331b detected by the magnetometer 330 over time. The detected signal 331b represents the magnitude of the combination of the bias magnetic field and the unbiased magnetic field over time. As a result, the detected signal 331b includes a pulse 333b related to the magnetic fields produced by magnetic nanoparticles of the complex 365 as the complex moves past the magnetometer 330.

The bias field magnitude 336 could be determined and/or generated by a variety of methods and related to a variety of signals and/or factors such that a background magnetic field and/or some other unwanted signal or field is not detected by the magnetometer 330. In some examples, this could include performing a linear operation (e.g., averaging, subtraction, correlation, filtering), a nonlinear operation (e.g., nonlinear filtering, application of some probabilistic or clustering algorithm), or some other operation on the signals detected by the magnetometer 330, e.g., operating the bias coil 335 such that the generated bias magnetic field operates to reduce the signal detected by the magnetometer 330 using negative feedback. In another example, a second magnetometer (not shown) could be included (e.g., a magnetometer that is less-sensitive than, that has a greater dynamic range than, that detects magnetic fields at a different location then, or that is otherwise differently configured from the magnetometer 330) and the output of the second magnetometer could be used to determine the bias field magnitude.

A body-mountable device (e.g., 300) could include additional or alternative means for creating a bias magnetic field. For example, the body-mountable device 300 could include three or more coils configured to generate a bias magnetic field having a specified direction, magnitude, and/or other specified properties. In some examples, a permanent magnet or other magnetic materials (e.g., shims composed of mu-metal, ferrite, or some other magnetic material) could be configured to at least partially cancel an unwanted magnetic present at a magnetometer. In some examples, such magnetic elements could be actuated (e.g., motorized, have a modulatable magnetic property) such that the bias magnetic field produced can be controlled, e.g., based on a bias field magnitude determined based on an estimate of the unwanted magnetic field.

Magnetometers of embodiments described herein could be configured to detect magnetic fields produced intrinsically by magnetic nanoparticles, e.g., produced by permanently and/or spontaneously magnetic elements of the magnetic nanoparticles. Additionally or alternatively, the magnetic nanoparticles could be induced to produce a magnetic field, e.g., by being temporarily or permanently magnetized, by being exposed to an oscillating or otherwise time-varying electromagnetic field, or by some other means.

In some examples, a system could include an excitation coil (or some other antenna or other type of electromagnetic-field-producing element(s)) configured to produce an oscillating magnetic field in an environment of interest (e.g., in a portion of subsurface vasculature). The produced oscillating magnetic field could cause magnetic nanoparticles and/or other magnetic objects or materials in the environment of interest to produce a magnetic field that could be detected by a magnetometer positioned proximate to the environment of interest. One or more properties of the magnetic nanoparticles, analytes with which the magnetic nanoparticles are configured to selectively interact, and/or some other contents of the environment could be detected and/or determined based on the detected magnetic field. The magnetic field produced by the magnetic nanoparticles could include a reflected, phase-shifted, frequency-shifted, frequency-multiplied, or otherwise modified version of the field produced by the excitation coil. For example, the magnetic field produced by the magnetic nanoparticles could include a fundamental frequency at the frequency of the oscillating field produced by the excitation coil and a number of harmonics at frequencies that are multiples of the frequency of the oscillating field. A magnetometer configured to detect such a time-varying magnetic field produced by magnetic nanoparticles could include a SERF magnetometer, a SQUID, an inductive pickup (e.g., one or more coils of wire or otherwise-formed inductive antenna(s)), or some other time-varying magnetic field detecting means.

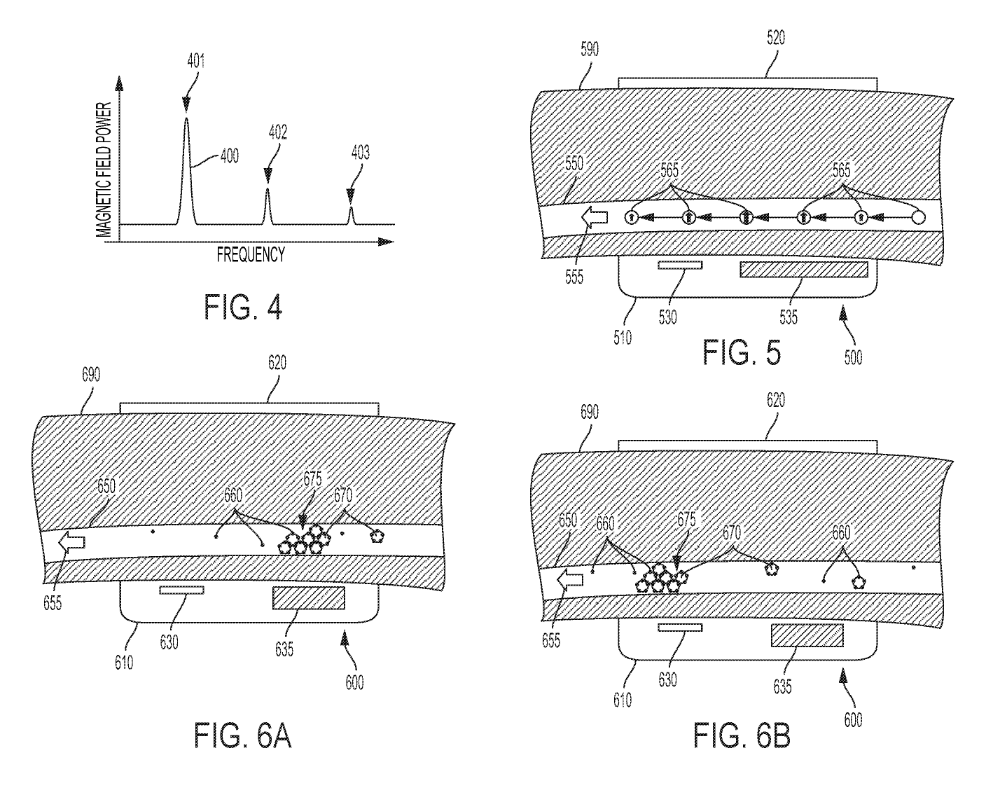

FIG. 4 shows an example power spectrum 400 of a magnetic field produced by magnetic nanoparticles in such a scenario. The magnetic field produced by the magnetic nanoparticles in response to the oscillating magnetic field produced by the excitation coil includes an oscillating field at substantially the same frequency as the frequency of the oscillating field produced by the excitation coil (the fundamental peak 401 of the power spectrum 400) and oscillating fields at multiples of the frequency of the oscillating field produced by the excitation coil (the harmonic peaks 402, 403 of the power spectrum 400). The presence, location, number, or other properties of magnetic nanoparticles proximate the magnetometer could be determined based on the amplitude, presence, phase shift, width, center frequency, or other properties of the harmonic peaks 402, 403, fundamental peak 401, and/or the aspects of the detected magnetic field corresponding to those peaks. In some examples, a filter or other means could be used to remove the fundamental peak 401 from the detected magnetic field to, e.g., increase a sensitivity of a detector to properties of the harmonic peaks 402, 403.

In some examples, a system could include a permanent magnet, an electromagnet, or some other means (e.g., some other magnetic flux source) configured to produce a magnetic field in an environment of interest sufficient to at least temporarily magnetize magnetic nanoparticles (e.g., ferromagnetic, superparamagnetic, or otherwise magnetic nanoparticles) in the environment of interest. A magnetic field produced by the magnetized magnetic nanoparticles could then be detected by a magnetometer and used to determine one or more properties of the magnetic nanoparticles and/or of an analyte with which the magnetic nanoparticles are configured to selectively interact.

FIG. 5 illustrates an example complex 565 that includes magnetic particles bound to an analyte of interest disposed in a blood vessel 550 (i.e., a portion of subsurface vasculature). The blood vessel 550 is located in an arm 590 and contains blood that is flowing (direction of flow indicated by the arrow 555). FIG. 5 illustrates the motion of the complex 565 in the blood vessel 550 over time in the direction of the flow 555. Arrows in the illustrated complex 565 over time indicate the degree of magnetization of the magnetic nanoparticles of the complex 565 over time. A body-mountable device 500 includes a housing 510 mounted outside of the blood vessel 550 by a mount 520 configured to encircle the arm 590. The body-mountable device 500 includes a magnetometer 530 disposed in the housing 510 and configured to detect magnetic fields at a location outside of the arm 590 (e.g., at a location within the magnetometer 530). The body-mountable device 500 additionally includes a magnetic flux source 535 (e.g., a permanent magnet, an electromagnet) disposed in the housing 510 and configured to produce a magnetic flux and/or field sufficient to at least partially magnetize and/or align a magnetic dipole of the magnetic nanoparticles of the complex 565. For example, the magnetic flux source 535 could be configured to produce a magnetic field in the blood vessel 550 that have a strength greater than approximately 100 Gauss.

As shown in FIG. 5, the complex 565 is moved for the blood flow 555 past the magnetic flux source 535. This can result in the magnetic nanoparticle(s) of the complex 565 becoming magnetized (illustrated by the increasing size of the arrows as the complex 565 passes over the magnetic flux source 535). The magnetometer 530 can then detect a magnetic field produced by the magnetized magnetic nanoparticles of the complex 565. The detected magnetic field could be used to determine one or more properties of the magnetic nanoparticles, the analyte, and/or the environment (e.g., the blood in the blood vessel 550). For example, a rate of reduction of the magnetization of the magnetic nanoparticles, a rate of rotation of the magnetic nanoparticles (in examples wherein the magnetic nanoparticles are aligned by the magnetic field produced by the magnetic flux source 535), or some other detected properties of the magnetic nanoparticles and/or the complex 565 could be detected and/or determined.

In some examples, magnetic nanoparticles and/or analytes bound to the magnetic nanoparticles in an environment could be collected such that a magnitude of the magnetic field produced by the magnetic particles and detected by a magnetometer is increased, e.g., to improve a determination of a property of the analyte by, e.g., increasing a magnitude of the detected magnetic field. FIGS. 6A and 6B illustrate, during respective first and second periods of time, example magnetic particles 660 and an analyte of interest 670 with which the magnetic particles 670 are configured to selectively interact disposed in a blood vessel 650 (i.e., a portion of subsurface vasculature). The blood vessel 650 is located in an arm 690 and contains blood that is flowing (direction of flow indicated by the arrow 655). A body-mountable device 600 includes a housing 610 mounted outside of the blood vessel 650 by a mount 620 configured to encircle the arm 690. The body-mountable device 600 includes a magnetometer 630 disposed in the housing 610 and configured to detect a magnetic field at a location outside of the arm 690 (e.g., at a location within the magnetometer 630). The body-mountable device 600 additionally includes a collection magnet 635 (e.g., a permanent magnet, an electromagnet) configured to exert an attractive magnetic force on the magnetic nanoparticles 660 such that at least some of the magnetic nanoparticles 670 in the blood vessel 650 are collected proximate the collection magnet 635. In the example shown in FIGS. 6A and 6B, this includes collecting magnetic nanoparticle 660 that are bound to instances of the analyte 670 into a bolus 675 located proximate the collection magnet 635.

FIG. 6A shows the body-mountable device 600 during a first period of time during which the collection magnet 635 is exerting an attractive magnetic force to attract magnetic nanoparticles 660 and instances of the analyte 670 bound thereto to form a bolus 675 of collected magnetic nanoparticles 660. FIG. 6B shows the body-mountable device 600 during a second period of time. The collection magnet 635 is configured and/or operated during the second period of time to exert a lesser magnetic force (e.g., to exert substantially no magnetic force) on the magnetic nanoparticles 660 such that the bolus 675 is released from the proximity of the collection magnet 635 and flows within the blood vessel 650 to a downstream location, past the magnetometer 630. The magnetometer 630 operates to detect a magnetic field produced by the magnetic nanoparticles 660 (e.g., by magnetic nanoparticles of the bolus 675) to determine a property of the magnetic nanoparticles 660, the analyte 670, and/or the bolus 675. For example, a number of instances of the analyte 670 in the bolus 675 (and/or a concentration or number of the analyte 670 in the blood overall) could be determined based on a magnitude or other properties of the detected magnetic field.

Note that the configuration and operation shown in FIGS. 6A and 6B are non-limiting examples. In some embodiments, a collection magnet could be collocated with a magnetometer (e.g., could act to collect magnetic nanoparticles proximate the magnetometer). In some examples, the magnetometer could operate to detect the magnetic field produced by the magnetic nanoparticles while the collection magnet is exerting an attractive magnetic force to collect the magnetic nanoparticles (e.g., by introducing a bias magnetic field using a coil or other magnetic materials to cancel the magnetic field generated by the collection magnet that is detected by the magnetometer, by configuring the magnetometer to detect magnetic fields in a direction perpendicular to a field produced by a collection magnet, by detecting oscillating magnetic fields produced by the magnetic nanoparticles, e.g., in response to exposure to an oscillating magnetic field produced by an excitation coil).

Magnetometers, devices containing magnetometers, magnetic nanoparticles, and other aspects and embodiments described herein (e.g., 100, 200, 300, 400, 500, 600) could be configured and/or operated to provide a variety of applications. In some examples, magnetic nanoparticles could be configured to bind to an analyte of interest, and one or more magnetometers could detect a magnetic field produced by the magnetic nanoparticles to determine one or more properties (e.g., a presence, a location, a number, a concentration) of the analyte. In some examples, a device could be configured to collect, release, separate, modify, or otherwise manipulate the magnetic nanoparticles to enable the detection, extraction, modification, or other manipulation of the analyte. Additionally or alternatively, the system could include an energy emitter and the energy emitter could emit energy toward collected magnetic nanoparticles and/or when it is detected that the analyte is present to alter one or more properties of the analyte (e.g., to destroy, denature, heat, change a conformation state of, other otherwise modify the analyte). In some examples, detection of one or more properties of an analyte bound to magnetic nanoparticles could enable the determination of a course of medical treatment, the adjustment of a dosage of a drug, the generation of a medical alert, or some other action. Other configurations, operations, and applications of the embodiments described herein are anticipated.

The terms “binding”, “bound”, and related terms used herein are to be understood in their broadest sense to include any interaction between the receptor and the target or another functionalized particle such that the interaction allows the target to be modified or destroyed by energy emitted from a wearable device.

### III. EXAMPLE WEARABLE DEVICES

Wearable devices as described herein can be configured to be mounted to an external body surface of a wearer and to enable a variety of applications and functions including the detection of magnetic fields produced by magnetic nanoparticles disposed in the body of the wearer (e.g., disposed in a portion of subsurface vasculature of the wearer). One or more magnetometers of the wearable device could be configured to detect the magnetic fields produced by magnetic nanoparticles disposed proximate the one or more magnetometers, as described elsewhere herein. Such wearable devices could enable a variety of applications, including measuring properties of the magnetic nanoparticles and/or an analyte with which the magnetic nanoparticles are configured to selectively interact (e.g., bind to), to detect other physiological information about a wearer (e.g., heart rate), indicating such measured information or other information to the wearer (e.g., using a vibrator, a screen, a beeper), or other functions.

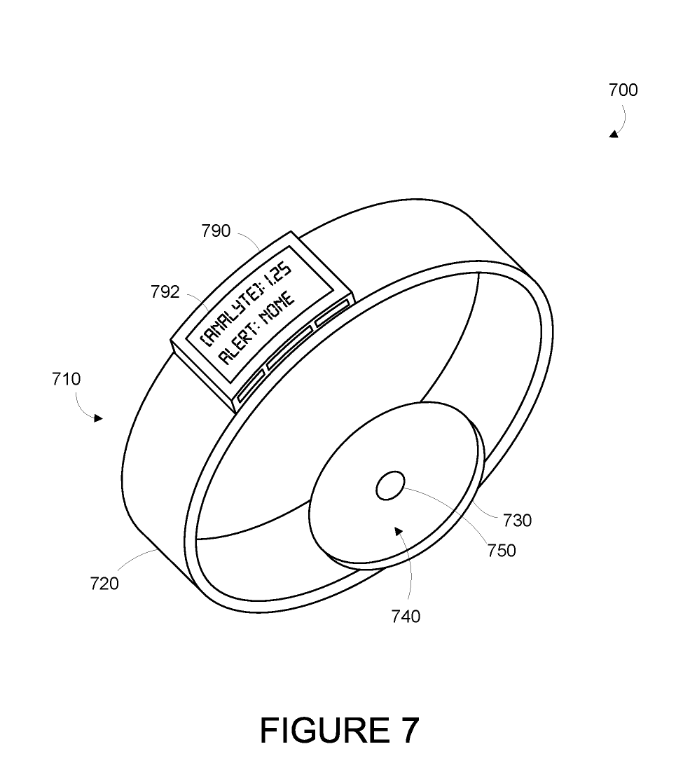

A wearable device 700 (illustrated in FIG. 7) can be configured to detect magnetic fields produced by magnetic nanoparticles disposed in a wearer's body (e.g., disposed in portions of subsurface vasculature proximate the device 700) or other physiological parameters of a person wearing the device. The term “wearable device,” as used in this disclosure, refers to any device that is capable of being worn at, on or in proximity to a body surface, such as a wrist, ankle, waist, chest, or other body part. In order to take in vivo measurements in a non-invasive manner from outside of the body, the wearable device may be positioned on a portion of the body where subsurface vasculature or other targets or elements of the body of the wearer are easily observable, the qualification of which will depend on the type of detection system used. The device may be placed in close proximity to the skin or tissue. A mount 710, such as a belt, wristband, ankle band, etc. can be provided to mount the device at, on or in proximity to the body surface. The mount 710 may prevent the wearable device from moving relative to the body to reduce measurement error and noise. In one example, shown in FIG. 7, the mount 710, may take the form of a strap or band 720 that can be worn around a part of the body. Further, the mount 710 may be an adhesive substrate for adhering the wearable device 700 to the body of a wearer.

A housing 730 is disposed on the mount 710 such that it can be positioned on the body. A contact surface 740 of the housing 730 is intended to be mounted facing to the external body surface. The housing 730 may include a magnetometer 750 for detecting magnetic field produced by magnetic nanoparticles disposed in the body of the wearer (e.g., magnetic nanoparticles disposed in portions of subsurface vasculature). The housing 730 could be configured to be water-resistant and/or water-proof. That is, the housing 730 could be configured to include sealants, adhesives, gaskets, welds, transparent windows, apertures, press-fitted seams, and/or other joints such that the housing 730 was resistant to water entering an internal volume or volumes of the housing 730 when the housing 730 is exposed to water. The housing 730 could further be water-proof, i.e., resistant to water entering an internal volume or volumes of the housing 730 when the housing 730 is submerged in water. For example, the housing 730 could be water-proof to a depth of 1 meter, i.e., configured to resist water entering an internal volume or volumes of the housing 730 when the housing 730 is submerged to a depth of 1 meter.

The magnetometer 750 is configured to detect a magnetic field produced by magnetic nanoparticles disposed proximate the magnetometer (e.g., within from approximately 1 millimeter to approximately 1 centimeter) in an environment of interest, e.g., a portion of subsurface vasculature of a wearer. The magnetometer could be configured to have a sensitivity such that the magnetometer can detect changes in a measured magnetic field of less than approximately 10 femtoteslas. The magnetometer could be configured to detect a direction, magnitude, property of change over time, or some other property of the magnetic fields produced by the magnetic nanoparticles.

The wearable device 700 could include one or more bias coils, magnets, shims, magnetic shielding elements, or other components to reduce a background magnetic field to which the magnetometer 750 is exposed (e.g., to cancel the effects of the Earth's magnetic field on the magnetometer 750) and/or to provide some other functionality.

The magnetometer 750 could be configured to detect an oscillating or otherwise time-varying magnetic field produced by the magnetic nanoparticles in response to exposure to an oscillating magnetic field produced by an excitation coil or other component (e.g., antenna) of the wearable device 700. In some examples, this could include the magnetometer including one or more inductive pickup coils configured to detect the produced oscillating or otherwise time-varying magnetic fields and/or to emit the oscillating magnetic field produced by the wearable device 700 (i.e., the excitation coil used to produce the oscillating magnetic field in the environment of interest is also part of the magnetometer and used to detect the oscillating or otherwise time-varying magnetic fields responsively produced by the magnetic nanoparticles).

The magnetometer could include a variety of components configured in a variety of ways to detect one or more properties of a magnetic field produced by magnetic nanoparticles. The magnetometer could include a superconducting quantum interference device (SQUID), spin-exchange relaxation-free (SERF) magnetometer, one or more inductive loops or coils or other antenna structures, a spin precession magnetometer, or some other magnetic-field-detecting components or devices. In examples wherein the magnetometer 750 includes elements having a very high temperature (e.g., an alkali vapor cell of a SERF) or a very low temperature (e.g., the Josephson junction(s) of a SQUID), the magnetometer 750 and/or the housing 710 could include means for insulating the high- or low-temperature elements or for otherwise controlling the temperature of such elements and/or preventing injury to a user due to exposure to extreme temperatures of such elements. For example, an alkali vapor cell and/or other laments of a SERF magnetometer could be wholly or partially contained in an evacuated volume (e.g., a dewar), insulated with an aerogel, or otherwise insulated.

The wearable device 700 may also include a user interface 790 via which the wearer of the device may receive one or more recommendations or alerts generated either from a remote server or other remote computing device, or from a processor within the device. The alerts could be any indication that can be noticed by the person wearing the wearable device. For example, the alert could include a visual component (e.g., textual or graphical information on a display), an auditory component (e.g., an alarm sound), and/or tactile component (e.g., a vibration). Further, the user interface 790 may include a display 792 where a visual indication of the alert or recommendation may be displayed. The display 792 may further be configured to provide an indication of the measured magnetic field and/or one or more determined properties of the magnetic nanoparticles and/or an analyte in the body of the wearer.

Note that example devices herein are configured to be mounted to a wrist of a wearer. However, the embodiments described herein could be applied to other body parts (e.g., an ankle, a thigh, a chest, a forehead, a thigh, a finger), or to detect magnetic fields produced by magnetic nanoparticles in other environments. For example, embodiments described herein could be applied to detect one or more properties in a target environment (e.g., a natural environment, an environment of an industrial, pharmaceutical, or water treatment process).

Wearable devices and other embodiments as described herein can include a variety of components configured in a variety of ways. Devices described herein could include electronics including a variety of different components configured in a variety of ways to enable applications of the wearable device. The electronics could include controllers, amplifiers, switches, display drivers, touch sensors, wireless communications chipsets (e.g., Bluetooth radios or other radio transceivers and associated baseband circuitry to enable wireless communications between the wearable device and some other system(s)), or other components. The electronics could include a controller configured to operate one or more magnetometers and/or other sensors to detect a magnetic field and/or to detect some other properties of a wearer. The controller could include a processor configured to execute computer-readable instructions (e.g., program instructions stored in data storage of the wearable device) to enable applications of the wearable device. The electronics can include additional or alternative components according to an application of the wearable device.

Wearable devices as described herein could include one or more user interfaces. A user interface could include a display configured to present an image to a wearer and to detect one or more finger presses of a wearer on the interface. The controller or some other component(s) of the electronics could operate the user interface to provide information to a wearer or other user of the device and to enable the wearer or other user to affect the operation of the wearable device, to determine some property of the wearable device and/or of the wearer of the wearable device (e.g., a concentration of an analyte in the blood of the wearer determined based on a detected magnetic field and/or a health state of a wearer of the wearable device), or to provide some other functionality or application to the wearer and/or user. As one example, the wearer could press an indicated region of the user interface to indicate that the wearable device should begin logging detected medical information about the wearer. Other indicated information, changes in operation of the wearable device, or other functions and applications of the user interface are anticipated.

Note that the embodiments illustrated in the Figures are illustrative examples and not meant to be limiting. Alternative embodiments, including more or fewer components in alternative configurations are anticipated. A wearable device could include multiple housings or other such assemblies each containing some set of components to enable applications of such a wearable device. For example, a wearable device could include a first housing within which are disposed one or more magnetometers configured to detect magnetic fields produced by magnetic nanoparticles disposed in the wearer's body (e.g., within portions of subsurface vasculature of the wearer) and a second housing containing a user interface and electronics configured to operate the magnetometer(s) and to present information to and receive commands from a user of the wearable device. A wearable device could be configured to perform a variety of functions and to enable a variety of applications. Wearable devices could be configured to operate in concert with other devices or systems; for example, wearable devices could include a wireless communication interface configured to transmit data indicative of one or more properties of the body of a wearer of the wearable device. Other embodiments, operations, configurations, and applications of a wearable device as described herein are anticipated.

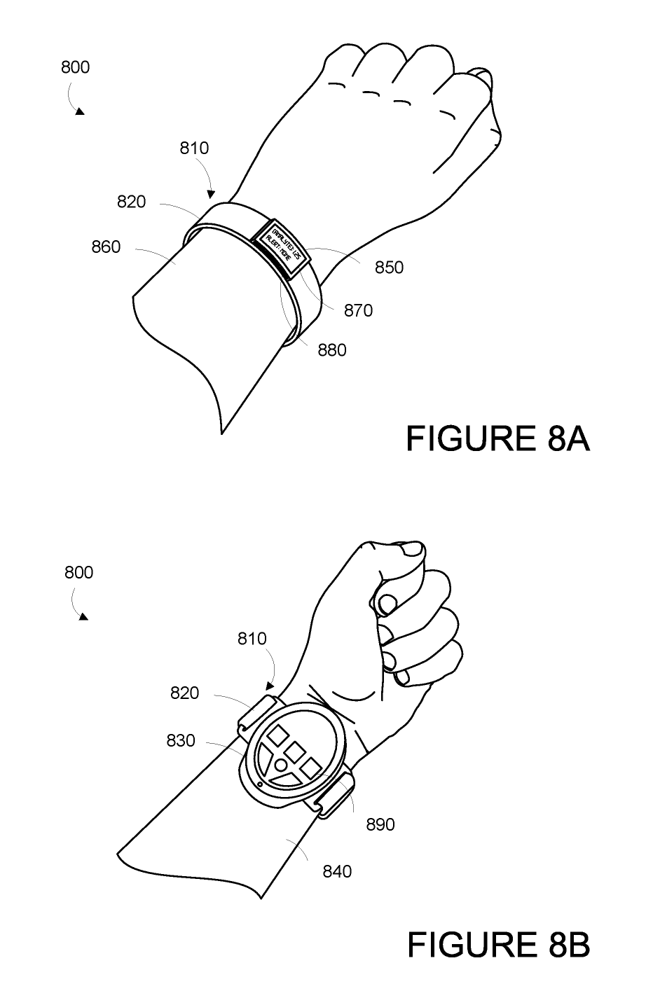

In some examples, the wearable device is provided as a wrist-mounted device, as shown in FIGS. 8A and 8B. The wrist-mounted device may be mounted to the wrist of a living subject with a wristband or cuff, similar to a watch or bracelet. As shown in FIGS. 8A and 8B, the wrist mounted device 800 may include a mount 810 in the form of a wristband 820, a housing 830 containing a data collection system and positioned on the anterior side 840 of the wearer's wrist, and a user interface 850 positioned on the posterior side 860 of the wearer's wrist. The wearer of the device may receive, via the user interface 850, one or more recommendations or alerts generated either from a remote server or other remote computing device, or alerts from the measurement platform. Such a configuration may be perceived as natural for the wearer of the device in that it is common for the posterior side 860 of the wrist to be observed, such as the act of checking a wrist-watch. Accordingly, the wearer may easily view a display 870 on the user interface. Further, the housing 830 may be located on the anterior side 840 of the wearer's wrist where the subsurface vasculature or other elements of the body of the wearer may be readily observable. However, other configurations are contemplated.

The display 870 may be configured to display a visual indication of the alert or recommendation and/or an indication of a measured magnetic field and/or some other property determined based on a detected magnetic field. Further, the user interface 850 may include one or more buttons 880 for accepting inputs from the wearer. For example, the buttons 880 may be configured to change the text or other information visible on the display 870. As shown in FIG. 8B, housing 830 may also include one or more buttons 890 for accepting inputs from the wearer. The buttons 890 may be configured to accept inputs for controlling aspects of the data collection system, such as initiating a measurement period, or inputs indicating the wearer's current health state (i.e., normal, migraine, shortness of breath, heart attack, fever, “flu-like” symptoms, food poisoning, etc.).

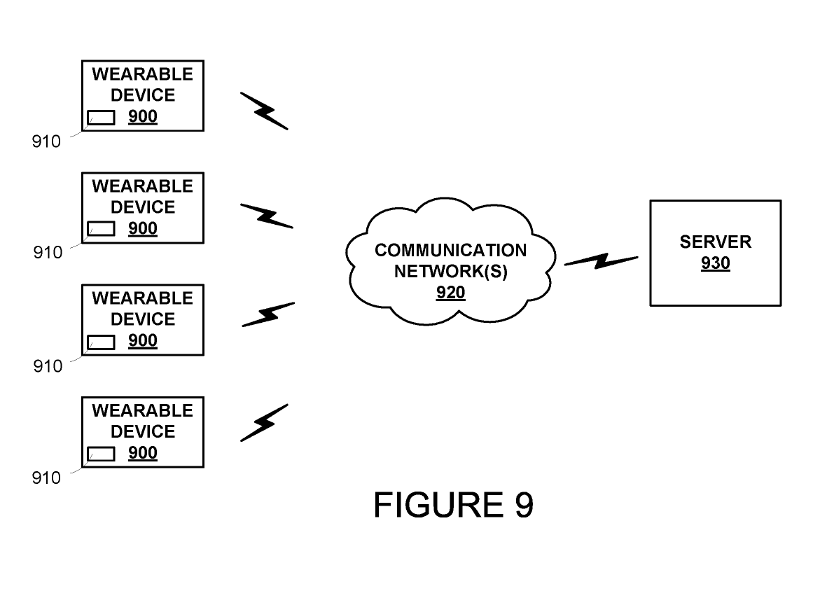

FIG. 9 is a simplified schematic of a system including one or more wearable devices 900. The one or more wearable devices 900 may be configured to transmit data via a communication interface 910 over one or more communication networks 920 to a remote server 930. In one embodiment, the communication interface 910 includes a wireless transceiver for sending and receiving communications to and from the server 930. In further embodiments, the communication interface 910 may include any means for the transfer of data, including both wired and wireless communications. For example, the communication interface may include a universal serial bus (USB) interface or a secure digital (SD) card interface. Communication networks 620 may be any one of may be one of: a plain old telephone service (POTS) network, a cellular network, a fiber network and a data network. The server 930 may include any type of remote computing device or remote cloud computing network. Further, communication network 920 may include one or more intermediaries, including, for example wherein the wearable device 900 transmits data to a mobile phone or other personal computing device, which in turn transmits the data to the server 930.

In addition to receiving communications from the wearable device 900, such as detected magnetic fields produced by magnetic nanoparticles disposed in a body of a wearer (e.g., disposed in portion(s) of subsurface vasculature of a wearer) and/or information determined therefrom (e.g., information about an analyte with which the magnetic nanoparticles are configured to selectively interact) or other collected physiological properties and data, the server may also be configured to gather and/or receive either from the wearable device 900 or from some other source, information regarding a wearer's overall medical history, environmental factors and geographical data. For example, a user account may be established on the server for every wearer that contains the wearer's medical history. Moreover, in some examples, the server 930 may be configured to regularly receive information from sources of environmental data, such as viral illness or food poisoning outbreak data from the Centers for Disease Control (CDC) and weather, pollution and allergen data from the National Weather Service. Further, the server may be configured to receive data regarding a wearer's health state from a hospital or physician. Such information may be used in the server's decision-making process, such as recognizing correlations and in generating clinical protocols.

Additionally, the server may be configured to gather and/or receive the date, time of day and geographical location of each wearer of the device during each measurement period. Such information may be used to detect and monitor spatial and temporal spreading of diseases. As such, the wearable device may be configured to determine and/or provide an indication of its own location. For example, a wearable device may include a GPS system so that it can include GPS location information (e.g., GPS coordinates) in a communication to the server. As another example, a wearable device may use a technique that involves triangulation (e.g., between base stations in a cellular network) to determine its location. Other location-determination techniques are also possible.

The server may also be configured to make determinations regarding the efficacy of a drug or other treatment based on information regarding the drugs or other treatments received by a wearer of the device and, at least in part, the detected magnetic field data and the indicated health state of the user. From this information, the server may be configured to derive an indication of the effectiveness of the drug or treatment. For example, if a drug is intended to treat nausea and the wearer of the device does not indicate that they are experiencing nausea after beginning a course of treatment with the drug, the server may be configured to derive an indication that the drug is effective for that wearer. In another example, a wearable device may be configured to detect cancer cells by detecting properties of magnetic nanoparticles that are configured to selectively interact with cancer cells. If a wearer is prescribed a drug intended to destroy cancer cells, but the server receives data from the wearable device indicating that the number of cancer cells in the wearer's blood has been increasing over a certain number of measurement periods, the server may be configured to derive an indication that the drug is not effective for its intended purpose for this wearer.

Further, some embodiments of the system may include privacy controls which may be automatically implemented or controlled by the wearer of the device. For example, where a wearer's collected magnetic field data and health state data are uploaded to a cloud computing network for trend analysis by a clinician, the data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined.

Additionally or alternatively, wearers of a device may be provided with an opportunity to control whether or how the device collects information about the wearer (e.g., information about a user's medical history, social actions or activities, profession, a user's preferences, or a user's current location), or to control how such information may be used. Thus, the wearer may have control over how information is collected about him or her and used by a clinician or physician or other user of the data. For example, a wearer may elect that data, such as health state and detected magnetic field data, collected from his or her device may only be used for generating an individual baseline and recommendations in response to collection and comparison of his or her own data and may not be used in generating a population baseline or for use in population correlation studies.

### IV. EXAMPLE ELECTRONICS PLATFORM FOR A DEVICE

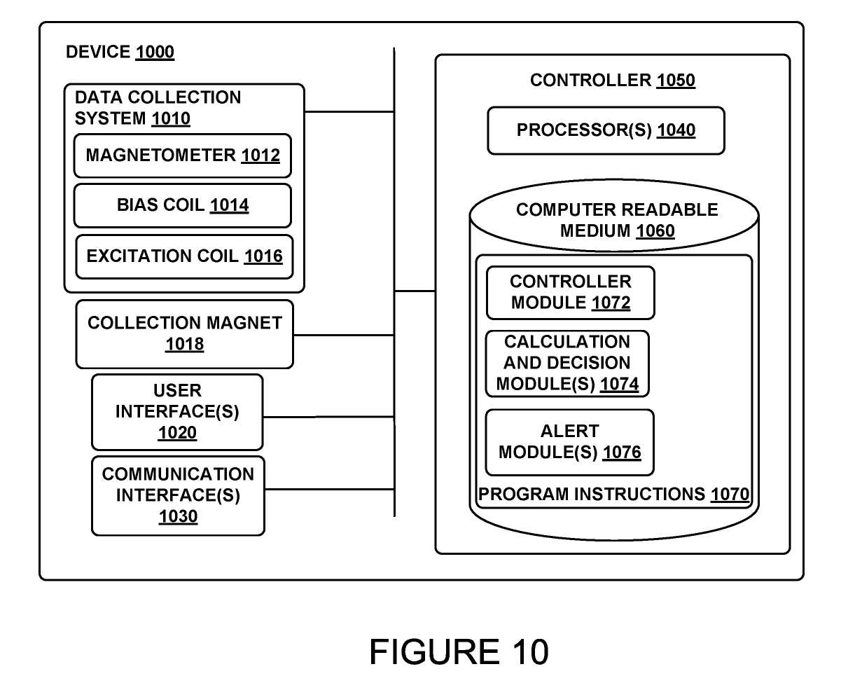

FIG. 10 is a simplified block diagram illustrating the components of a device 1000, according to an example embodiment. Device 1000 may take the form of or be similar to one of the wearable devices 100, 200, 300, 400, 500, 600, 700, or 800 shown in FIGS. 1, 2, 3, 4, 5, 6A, 6B, 7 and 8A-B. However, device 1000 may also take other forms, such as an ankle, waist, or chest-mounted device. Device 1000 could also take the form of a device that is not configured to be mounted to a body. For example, device 1000 could take the form of a handheld device configured to be maintained in proximity to an environment of interest (e.g., a body part, a biological sample container, a volume of a water treatment system) by a user or operator of the device 1000 or by a frame or other supporting structure. In some examples, device 1000 could be or could form part of device configured to detect properties of an ex vivo and/or in vitro environment (e.g., the device 1000 could be configured to be operated as part of a flow cytometry experiment). Device 1000 also could take other forms.

In particular, FIG. 10 shows an example of a device 1000 having a data collection system 1010 that includes a magnetometer 1012, a bias coil 1014, and an excitation coil 1016, a collection magnet 1018, a user interface 1020, communication interface 1030 for transmitting data to a remote system, and a controller 1050. The components of the device 1000 may be disposed on a mount or on some other structure for mounting the device to enable stable detection of one or more properties (e.g., magnetic fields produced by magnetic nanoparticles) of an environment of interest (e.g., of a body of a wearer of the device 1000), for example, mounting to an external body surface where one or more portions of subsurface vasculature or other anatomical elements are readily observable.

Controller 1050 may be provided as a computing device that includes one or more processors 1040. The one or more processors 1040 can be configured to execute computer-readable program instructions 1070 that are stored in the computer readable data storage 1060 and that are executable to provide the functionality of a device 1000 described herein.

The computer readable medium 1060 may include or take the form of one or more non-transitory, computer-readable storage media that can be read or accessed by at least one processor 1040. The one or more computer-readable storage media can include volatile and/or non-volatile storage components, such as optical, magnetic, organic or other memory or disc storage, which can be integrated in whole or in part with at least one of the one or more processors 1040. In some embodiments, the computer readable medium 1060 can be implemented using a single physical device (e.g., one optical, magnetic, organic or other memory or disc storage unit), while in other embodiments, the computer readable medium 1060 can be implemented using two or more physical devices.

The magnetometer 1012 is configured to detect a magnetic field produced by magnetic nanoparticles disposed proximate the magnetometer (e.g., within from approximately 1 millimeter to approximately 1 centimeter) in an environment of interest, e.g., a portion of subsurface vasculature of a wearer. The magnetometer could be configured to have a sensitivity such that the magnetometer can detect changes in a measured magnetic field of less than approximately 10 femtoteslas. The magnetometer could include one or more inductive pickup coils configured to detect an oscillating or otherwise time-varying magnetic field produced by the magnetic nanoparticles in response to exposure to an oscillating magnetic field produced by the excitation coil 1016 or some other component (e.g., antenna) of the device 1000. The magnetometer could include amplifiers, oscillators, ADCs, switches, filters, light emitter, light detectors, or other components configured to detect a magnetic field using one or more magnetic-field-sensitive elements of the magnetometer 1012. For example, the magnetometer 1012 could be a SERF magnetometer that includes an alkali vapor cell (i.e., an enclosed volume containing a high-pressure, high-temperature vapor that includes alkali metal atoms) and the electronics could include a heater configured to vaporize the alkali metal in the vapor cell, a pump laser configured to emit circularly polarized light into the vapor cell to align the alkali metal atoms, a probe laser configured to probe the aligned alkali atoms with linearly polarized light, and a light detector configured to detect the change in orientation of the linearly polarized light that is related to the detected magnetic field. Other examples of magnetometers and electronics thereof are anticipated.

The bias coil 1014 is configured to produce a bias magnetic field to reduce a background magnetic field to which the magnetometer 1012 is exposed (e.g., to cancel the effects of the Earth's magnetic field on the magnetometer 1012) and/or to provide some other functionality. The bias coil 1014 could be driven according to a bias field magnitude determined based on an output of the magnetometer 1012, an output of some other magnetometer (not shown), an output of an accelerometer, gyroscope, or some other sensor, or based on some other consideration.

The collection magnet 1018 is configured to produce an attractive magnetic force sufficient to collect magnetic nanoparticles proximate the device 1000 (e.g., proximate the magnetometer 1012). The collection magnet 1018 could be a permanent magnet and/or an electromagnet. In some examples, the collection magnet 1018 could be operated to collect magnetic nanoparticles (e.g., by exerting an attractive magnetic force) during a first period of time and subsequently to release the collected magnetic nanoparticles (e.g., to allow detection, by the magnetometer 1012, of a magnetic field produced by the collected magnetic nanoparticles). Further, the collection magnet 1018 and/or some other magnetic element(s) of the device 100 could be configured to permanently or temporarily magnetize the magnetic nanoparticles in an environment of interest (e.g., in a portion of subsurface vasculature).

Note that a device could include a subset of the elements illustrated here, e.g., a device could lack a bias coil, excitation coil, collection magnet, and/or some other combination of elements. Further, a device could include multiple of one or more illustrated elements. For example, a device could include multiple magnetometers configured to detect a magnetic field at respective multiple different locations and/or in multiple different directions. In another example, a device could include multiple bias coils to cancel magnetic fields in multiple different directions and/or for multiple different magnetometers. In some examples, multiple illustrated elements of the device 1000 could be implemented as the same component and/or share some component(s) in common. For example, the excitation coil 1016 could form part of the magnetometer 1012 and could be used to detect an oscillating or otherwise time-varying magnetic field produced by the magnetic nanoparticles in response to exposure to an oscillating magnetic field produced by the excitation coil 1016.

The program instructions 1070 stored on the computer readable medium 1060 may include instructions to perform any of the methods described herein. For instance, in the illustrated embodiment, program instructions 1070 include a controller module 1072, calculation and decision module 1074 and an alert module 1076.

Calculation and decision module 1074 may include instructions for operating the magnetometer 1012, bias coil 1014, and/or excitation coil 1016 to detect magnetic fields produced by magnetic nanoparticles proximate the magnetometer 1012 and analyzing data generated by the magnetometer 1012 to determine information about magnetic nanoparticles and/or analytes in a body (e.g., by detecting pulses related to aggregates of magnetic nanoparticles in the change of a detected magnetic field over time) or other information (e.g., health states) of a body of a wearer of the device 1000, such as a concentration of an analyte in blood of the body at a plurality of points in time. Calculation and decision module 1074 can additionally include instructions for analyzing the data to determine if a medical condition or other specified condition is indicated, or other analytical processes relating to the environment proximate to the device 1000. In particular, the calculation and decision module 1074 may include instructions for operating the bias coil 1014 to reduce a magnetic field detected by the magnetometer 1012 and/or instructions for operating the excitation coil 1016 to produce an oscillating or otherwise time-varying magnetic field in an environment containing magnetic nanoparticles. These instructions could be executed at each of a set of preset measurement times.

The controller module 1072 can also include instructions for operating a user interface 1020. For example, controller module 1072 may include instructions for displaying data collected by the data collection system 1010 and analyzed by the calculation and decision module 1074, or for displaying one or more alerts generated by the alert module 1076. Controller module 1072 may include instructions for displaying data related to a detected magnetic field produced by magnetic nanoparticles in one or more portions of subsurface vasculature or some other detected and/or determined health state of a wearer. Further, controller module 1072 may include instructions to execute certain functions based on inputs accepted by the user interface 1020, such as inputs accepted by one or more buttons disposed on the user interface.

Communication interface 1030 may also be operated by instructions within the controller module 1072, such as instructions for sending and/or receiving information via a wireless antenna, which may be disposed on or in the device 1000. The communication interface 1030 can optionally include one or more oscillators, mixers, frequency injectors, etc. to modulate and/or demodulate information on a carrier frequency to be transmitted and/or received by the antenna. In some examples, the device 1000 is configured to indicate an output from the processor by modulating an impedance of the antenna in a manner that is perceivable by a remote server or other remote computing device.

The program instructions of the calculation and decision module 1074 may, in some examples, be stored in a computer-readable medium and executed by a processor located external to the device 1000. For example, the device 1000 could be configured to collect certain data regarding magnetic fields produced by magnetic nanoparticles disposed in the body of the user and then transmit the data to a remote server, which may include a mobile device, a personal computer, the cloud, or any other remote system, for further processing.

The computer readable medium 1060 may further contain other data or information, such as medical and health history of a user of the device 1000, that may be useful in determining whether a medical condition or some other specified condition is indicated. Further, the computer readable medium 1060 may contain data corresponding to certain physiological parameter baselines, above or below which a medical condition is indicated. The baselines may be pre-stored on the computer readable medium 1060, may be transmitted from a remote source, such as a remote server, or may be generated by the calculation and decision module 1074 itself. The calculation and decision module 1074 may include instructions for generating individual baselines for the user of the device 1000 based on data collected over a certain number of measurement periods. Baselines may also be generated by a remote server and transmitted to the device 1000 via communication interface 1030. The calculation and decision module 1074 may also, upon determining that a medical or other emergency condition is indicated, generate one or more recommendations for the user of the device 1000 based, at least in part, on consultation of a clinical protocol. Such recommendations may alternatively be generated by the remote server and transmitted to the device 1000.

In some examples, the collected magnetic field data, baseline profiles, health state information input by device users and generated recommendations and clinical protocols may additionally be input to a cloud network and be made available for download by a user's physician. Trend and other analyses may also be performed on the collected data, such as analyte and/or magnetic nanoparticle data and health state information, in the cloud computing network and be made available for download by physicians or clinicians.

Further, detected magnetic field data and determined magnetic nanoparticle, analyte, and health state data from individuals or populations of device users may be used by physicians or clinicians in monitoring efficacy of a drug or other treatment. For example, high-density, real-time data may be collected from a population of device users who are participating in a clinical study to assess the safety and efficacy of a developmental drug or therapy. Such data may also be used on an individual level to assess a particular wearer's response to a drug or therapy. Based on this data, a physician or clinician may be able to tailor a drug treatment to suit an individual's needs.

In response to a determination by the calculation and decision module 1074 that a medical or other specified condition is indicated, the alert module 1076 may generate an alert via the user interface 1020. The alert may include a visual component, such as textual or graphical information displayed on a display, an auditory component (e.g., an alarm sound), and/or tactile component (e.g., a vibration). The textual information may include one or more recommendations, such as a recommendation that the user of the device contact a medical professional, seek immediate medical attention, or administer a medication.

### V. EXAMPLE METHODS

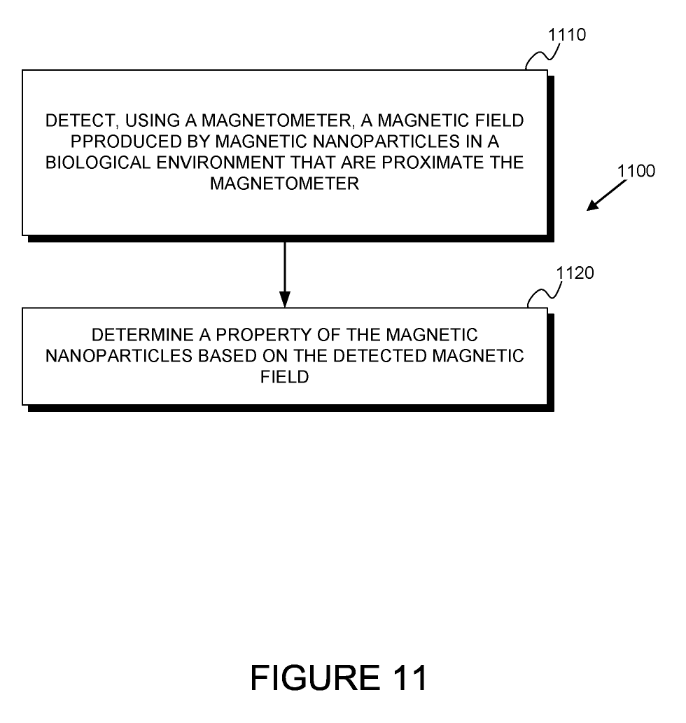

FIG. 11 is a flowchart of an example method 1100 for detecting properties of magnetic nanoparticles in a biological environment by detecting a magnetic field produced by the magnetic nanoparticles. The method 1100 includes detecting, using a magnetometer, a magnetic field produced by magnetic particles in a biological environment that are proximate the magnetometer (1110). This could include detecting a magnitude, direction, magnitude in a particular direction, a pattern or property of change over time of a property of the produced magnetic field, or some other property of the produced magnetic field. In some examples, detecting the magnetic field produced by the magnetic nanoparticles (1110) could include detecting the produced field at more than one location proximate to more than one magnetometer. Detecting the magnetic field produced by the magnetic nanoparticles (1110) could include producing an oscillating magnetic field in the biological environment and detecting a time-varying magnetic field responsively reflected, phase-shifted, frequency-shifter, frequency-multiplied, or otherwise produced by the magnetic nanoparticles. Detecting the magnetic field produced by the magnetic nanoparticles (1110) could include applying a bias magnetic field (e.g., by operating a bias coil disposed proximate the magnetometer) to cancel a background magnetic field (e.g., a magnetic field produced by the Earth) to which the magnetometer is exposed.

The method 1100 additionally includes determining a property of the magnetic nanoparticles based on the detected magnetic field (1120). This could include determining the orientation and/or location of one or more of the magnetic nanoparticles, a degree of aggregation of the magnetic nanoparticles, or the detection of some other property of the magnetic nanoparticles. Determining a property of the magnetic nanoparticles (1120) could include determining and/or detecting features of the detected magnetic field, e.g., detecting the amplitude, width, timing, or other properties of pulses in the detected magnetic field produced by the magnetic nanoparticles over time. Further, such determined properties of the magnetic nanoparticles could be related to properties of an analytes of interest with which the magnetic nanoparticles are configured to selectively interact (e.g., to bind to). For example, multiple magnetic nanoparticles could bind to a single instance of an analyte (e.g., to a single cancer cell) such that detection of an aggregate of magnetic nanoparticles (e.g., detection of a large amplitude magnetic field produced by such aggregated magnetic nanoparticles) allows for the determination that the single instance of the analyte is present (e.g., that a cancel cell is present in a portion of subsurface vasculature). Other properties of a detected magnetic field produced by magnetic nanoparticles could be used in similar or different ways to determine properties of one or more analytes in an environment of interest.

The method 1100 could include additional steps or elements. For example, the method 1100 could include introducing the magnetic particles into the biological environment (e.g., into a portion of subsurface vasculature by injecting, ingesting, transdermally transferring, or otherwise introducing the engineered particles into a lumen of vasculature of a human). In some examples, the method 1100 could include collecting the magnetic particles in a portion of subsurface vasculature, e.g., to extract the magnetic nanoparticles and/or to increase a magnitude of the magnetic field produced by the magnetic nanoparticles as detected by the magnetometer. The method 1100 could include additional or alternative steps.

### VI. CONCLUSION

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

While various aspects and embodiments herein are described in connection with detecting magnetic fields produced and/or influenced by magnetic nanoparticles disposed in particular example biological environments (e.g., a portion of subsurface vasculature) to detect and/or determine properties (e.g., a presence, a concentration, a number, a binding state) of the magnetic nanoparticles, other applications and environments are possible. Aspects and embodiments herein could be applied to detect properties of magnetic nanoparticles in in vivo or in vitro human or animal tissues, a fluid in a scientific, medical, or industrial testing process, or some other environment. Properties of magnetic nanoparticles disposed in a natural environment, e.g., a lake, river, stream, marsh, or other natural locale could be detected. Properties of magnetic nanoparticles disposed in a fluid environment of an industrial process or other artificial environment, e.g., a water treatment process, a food preparation process, a pharmaceutical synthesis process, a chemical synthesis process, a brewing and/or distilling process, or other artificial locale could be detected. Properties of magnetic nanoparticles disposed in an environment that includes a flowing fluid (e.g., fluid flowing in a blood vessel, a pipe, a culvert) and/or a substantially static fluid could be detected. Other environments and applications of aspects and embodiments described herein are anticipated.

Where example embodiments involve information related to a person or a device of a person, some embodiments may include privacy controls. Such privacy controls may include, at least, anonymization of device identifiers, transparency and user controls, including functionality that would enable users to modify or delete information relating to the user's use of a product.

Further, in situations wherein embodiments discussed herein collect personal information about users, or make use of personal information, the users may be provided with an opportunity to control whether programs or features collect user information (e.g., information about a user's medical history, social network, social actions or activities, profession, a user's preferences, or a user's current location), or to control whether and/or how to receive content from the content server that may be more relevant to the user. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over how information is collected about the user and used by a content server.

What is claimed is:

1. A device comprising:
a magnetometer, wherein the magnetometer is configured to be positioned proximate to a body, wherein the magnetometer is configured to detect magnetic fields produced by magnetic nanoparticles in the body that are proximate the magnetometer; and
a controller operably coupled to the magnetometer, wherein the controller comprises a computing device programmed to perform controller operations comprising:
operating the magnetometer to detect a magnetic field; and
determining a property of magnetic nanoparticles in the body based on the detected magnetic field.

2. The device of claim 1, wherein determining a property of magnetic nanoparticles in the body based on the detected magnetic field comprises determining a degree of aggregation of the magnetic nanoparticles in the body.

3. The device of claim 1, wherein the controller operations further comprise determining a property of an analyte bound to the magnetic nanoparticles based the determined property of the magnetic nanoparticles.

4. The device of claim 3, wherein determining a property of an analyte bound to the magnetic nanoparticles comprises determining an amount of the analyte in the body.

5. The device of claim 3, wherein the magnetometer being configured to be positioned proximate to the body comprises the magnetometer being configured to be positioned on an external body surface of the body.

6. The device of claim 1, further comprising:
a further magnetometer, wherein the further magnetometer is configured to be positioned proximate to the body, wherein the further magnetometer is configured to detect further magnetic fields produced by magnetic nanoparticles in the body that are proximate the further magnetometer, wherein the controller operations further comprise operating the further magnetometer to detect the further magnetic fields, and wherein determining the property of magnetic nanoparticles in the body comprises determining the property of magnetic nanoparticles in the body based on the further magnetic fields detected using the further magnetometer.

7. The device of claim 1, wherein the magnetometer comprises a spin-exchange relaxation-free atomic magnetometer.

8. The device of claim 1, wherein the magnetometer comprises a superconducting quantum interference device.

9. The device of claim 1, further comprising:
a magnetic flux source, wherein the magnetic flux source is configured to be positioned proximate to the body and to magnetize magnetic nanoparticles in the body that are proximate the magnetic flux source, and wherein operating the magnetometer comprises operating the magnetometer to detect magnetic fields produced by magnetic nanoparticles in the body that have been magnetized by the magnetic flux source.

10. The device of claim 1, further comprising:
a collection magnet, wherein the collection magnet is configured to be positioned proximate to the body, wherein the collection magnet is configured to exert an attractive magnetic force on magnetic nanoparticles in the body proximate to the collection magnet, and wherein the attractive magnetic force is sufficient to collect the magnetic nanoparticles proximate to the collection magnet.

11. The device of claim 1, further comprising an excitation coil, wherein the excitation coil is configured to be positioned proximate to the body and to produce an oscillating magnetic field in the body, and wherein operating the magnetometer comprises operating the magnetometer to detect time-varying magnetic fields produced by magnetic nanoparticles in the body in response to the oscillating magnetic field produced by the excitation coil.

12. The device of claim 1, further comprising:
at least one bias coil, wherein the at least one bias coil is configured to produce a bias magnetic field such that the magnetic field detected by the magnetometer is reduced by an amount related to the bias magnetic field, and wherein the controller operations further comprise:
determining a bias field magnitude; and
operating the at least one bias coil to produce the bias magnetic field according to the determined bias field magnitude.

13. A method comprising:
positioning a magnetometer on a body surface of a body;
detecting, using the magnetometer, a magnetic field produced by magnetic nanoparticles in the body that are proximate the magnetometer; and
determining a property of magnetic nanoparticles in the body based on the detected magnetic field.

14. The method of claim 13, wherein determining a property of magnetic nanoparticles in the body based on the detected magnetic field comprises determining a degree of aggregation of the magnetic nanoparticles in the body.

15. The method of claim 13, further comprising:
determining a property of an analyte bound to the magnetic nanoparticles based on the determined property of the magnetic nanoparticles.

16. The method of claim 15, wherein determining a property of an analyte bound to the magnetic nanoparticles comprises determining an amount of the analyte in the body.

17. The method of claim 13, further comprising:
producing an oscillating magnetic field in the body, wherein detecting a magnetic field proximate to the body comprises detecting a time-varying magnetic field produced by magnetic nanoparticles in the body in response to exposure to the produced oscillating magnetic field.

18. The method of claim 17, wherein detecting a time-varying magnetic field produced by magnetic nanoparticles in the body in response to exposure to the produced oscillating magnetic field comprises detecting a time-varying magnetic field at a frequency that is a multiple of the frequency of the produced oscillating magnetic field.

19. The method of claim 13, further comprising:
exerting, using a collection magnet, an attractive magnetic force on magnetic nanoparticles in the body proximate to the collection magnet, wherein the attractive magnetic force is sufficient to collect the magnetic nanoparticles proximate to the collection magnet.

20. The method of claim 13, further comprising:
detecting, using a further magnetometer, a further magnetic field proximate to a body, wherein detecting the further magnetic field proximate to a body comprises detecting a further magnetic field produced by magnetic nanoparticles in the body that are proximate the further magnetometer, and wherein determining the property of magnetic nanoparticles in the body comprises determining the property of magnetic nanoparticles in the body based on the further magnetic field detected using the further magnetometer.
